(19) United States
(12) Patent Application Publication
(10) Pub. No.: US 2026/0325584 A1
(43) Pub. Date: Oct. 22, 2026

(54) TREATMENT OF COMPLEX REGIONAL PAIN SYNDROME WITH EXTRACELLULAR VESICLE COMPOSITION

(71) Applicant: Direct Biologics, LLC, Austin, TX (US)

(72) Inventors: Timothy Alexander Moseley, Fallbrook, CA (US); Kenneth Allen Pettine, Fort Collins, CO (US); Richard Paicius, Newport Beach, CA (US)

(21) Appl. No.: 19/491,029

(22) PCT Filed: Jun. 7, 2024
(86) PCT No.: PCT/US2024/033022

§ 371 Date: Dec. 8, 2025

Related U.S. Application Data
(60) Provisional application No. 63/507,207, filed on Jun. 9, 2023.

(51) Int. Cl. A61K 35/28; A61P 25/00; A61P 29/02

(52) U.S. Cl. CPC A61K 35/28; A61P 25/00; A61P 29/02

(57) ABSTRACT

Disclosed are methods of treating complex regional pain syndrome in a subject by administering a therapeutic MSC secretome product made by a method comprising culturing bone marrow-derived MSCs under conditions that include oxygen tension below 5% and a culture media with a pH below 7.

### CROSS REFERENCE

This application claims the benefit of U.S. Provisional Application No. 63/507,207, filed on Jun. 9, 2023, which is incorporated herein by reference in its entirety.

### BACKGROUND

Complex regional pain syndrome (CRPS) is an extremely painful and disabling disorder that can occur in an extremity after a major trauma or surgery or may develop spontaneously. CRPS is a multi-system disease that incorporates different elements from the autonomic nervous system, however the largest driver of symptomatology is inflammation. Currently, there is no single proven effective treatment modality for patients suffering from CRPS identified from clinical trials. There exists a need for a safe and effective treatment for CRPS.

### SUMMARY

Disclosed herein is a method of treating complex regional pain syndrome (CRPS) in a subject, An aspect of the present disclosure is a method of treating complex regional pain syndrome (CRPS) in a subject in need thereof, the method comprising administering to the subject a composition comprising a therapeutic mesenchymal stem cell (MSC) secretome composition comprising extracellular vesicles, wherein at least 80% of the extracellular vesicles in the therapeutic MSC secretome composition are CD63⁺ CD9⁻ CD81⁻. In some embodiments, the subject has a decrease of at least 50% in a Visual Analog Scale of Pain (VAS) score after administration compared to before administration. Also disclosed herein is a method of treating complex regional pain syndrome (CRPS) in a subject in need thereof, the method comprising administering to the subject a composition comprising a therapeutic mesenchymal stem cell (MSC) secretome composition comprising extracellular vesicles, wherein the subject has a decrease of at least 50% in a Visual Analog Scale of Pain (VAS) score after administration compared to before administration. In some embodiments, at least 80% of the extracellular vesicles in the therapeutic MSC secretome composition are CD63+ CD9− CD81−. In some embodiments, the subject has a minimum VAS score of greater than 6 in an affected extremity prior to administration. In some embodiments, the subject has a decrease in a Brief Pain Inventory (BPI) score after administration compared to before administration. In some embodiments, the subject has a decrease in a BPI score of at least 10%, at least 20%, at least 30%, or greater than 30% after administration compared to before administration. In some embodiments, the subject has a decrease in a BPI pain interference score of at least 10%, at least 20%, at least 30%, or greater than 30% after administration compared to before administration. In some embodiments, the subject has an increase in a range of motion assessment score of at least 50%, at least 60%, at least 70%, at least 75%, at least 80%, at least 85%, at least 95%, or at least 100% after administration compared to before administration. In some embodiments, the subject has an increase in a Jamar dynamometer test assessment score after administration as compared to before administration. In some embodiments, the increase in the Jamar dynamometer test assessment score is in an upper extremity. In some embodiments, the subject has an equalization of grip strength after administration. In some embodiments, the subject has an increase in grip strength of at least 5 kg, at least 10 kg, at least 15 kg, at or least 20 kg after administration compared to before administration. In some embodiments, the subject has an increase in grip strength of at least 50%, at least 75%, at least 100%, at least 125%, at least 150%, at least 175%, at least 200%, or at least 210% after administration compared to before administration. In some embodiments, the subject has an increase in grip strength in an affected limb. In some embodiments, the subject has an increase in grip strength in a control limb. In some embodiments, the subject has an improvement after administration compared to before administration in one or more of: limb range of motion, joint range of motion, sudomotor changes, edema, allodynia, and hypesthesia. In some embodiments, the subject has an improvement in an Short Form 36 Health Survey Questionnaire (SF-36) Questionnaire score after administration compared to before administration. In some embodiments, the improvement in the SF-36 Questionnaire score comprises one or more of: an improvement in pain, an improvement in energy levels, an improvement in social functioning, an improvement in physical functioning, a positive trend in emotional wellbeing, and an improvement in general health. In some embodiments, the subject has chronic intractable pain of one or more upper or lower extremities. In some embodiments, the subject does not experience escalating or changing pain in the 30 days preceding administration. In some embodiments, the subject experiences decreased pain flare-ups, decreased swelling, and/or decreased burning sensations in an affected limb after administration compared to before administration. In some embodiments, the administration improves hair and/or nail growth in an affected limb of the subject. In some embodiments, the therapeutic MSC secretome composition further comprises one or more of the following proteins: Ferritin, NUP85, LAMP2, GPR115, Serpin F1, OPN, PAI-1, DAPP1, Cathepsin B, Semaphorin 6C, PDGF R alpha, Sortilin, Serpin B6, Dkk-3, Thrombomodulin, PF4, MIF, Periostin, Furin, TIMP-1, Decorin, PCK1, CD99, CD63, CD9, CD81, Transferrin, DcR3, Lumican, TIMP-2, SLITRK5, FAP, Artemin, DPPII, cIAP-1, Pentraxin 3, Visfatin, Neprilysin, Albumin, Galectin-1, UNC5H3, IL-20 R beta, SREC-IL, JAM-C, TNF RI, htPAPP-A, eNOS, MSP R, TPP1, LAMP1, B2M, NCAM-1, HIF-1 alpha, ST6GAL1, CD99-L2, Plexin A4, EMMPRIN, p53, Semaphorin 7A, NKp80, Cystatin B, Osteoadherin, Midkine, Calreticulin, Osteoactivin, Legumain, TAZ, Cathepsin L, RBP4, Serpin A4, JAM-A, MCSF, LIMPII, OPG, IL-22, Galectin-3, MOG, Trypsin 3, SIRP alpha, and Syndecan-4, and at least one protein selected from the following: Ferritin, IGFBP-4 IL-1 R6 GSTM1, NUP85, LAMP2, MeprinA, IL-1 F10, bIG-H3, GPR115, TGFb1, Ephrin-A4, CD109, Serpin F1, IGFBP-6, HS3ST4, Aminopeptidase LRAP, OPN, PAI-1, DAPP1, GDF-9, Cathepsin B, IGFBP-2, Semaphorin 6C, IGF-2, PDGF R alpha, Sortilin, Serpin B6, Dkk-3, CNTF, TSP-1, GM-CSF Ra, Thrombomodulin, Endoglycan, IGFBP-3, RGM-C, PF4, MIF, TGM4, Periostin, Furin, TIMP-1, PAPP-A, Decorin, PCK1, Arylsulfatase A, CD99, CA2, PRDX4, Transferrin, DcR3, GP73, LAIR2, ULBP-4, Lumican, TIMP-2, TFPI, SOX2, SLITRK5, FAP, Spinesin, ENPP-2, CD97, CTACK, Integrin alpha 1, EXTL3, IL-18 BPa, PD-L2, PSMA, IL-20 Ra, Glyoxalase II, Trypsin 1, IGF-2R, ADAMTSL-1, Erythropoietin, Plexin D1, DNMT3A, BCL-2, CL-P1, Ephrin-B3, FABP6, CHI3L1, FCRLS, TFF3, Artemin, DPPII, cIAP-1, PDGF Rb, Pentraxin 3, Angiotensinogen, Follistatin, CF VII, Persephin, TRAIL R1, THAP11, CD200, CLEC-2, AMIGO, IGFBP-5, PON1, SOX7, GALNT10, Visfatin, Progranulin, PCSK2, GKN1, IL-18, Neprilysin, Stabilin-2, IL-17 RD, Albumin, Follistatin-like 1, MMP-10, FKBP51, LRRC4, Pref-1, Galectin-1, Troponin C, UNC5H3, FLRT2, CD314, Semaphorin 6B, Netrin-4, CD27 Ligand, IL-20 R beta, Semaphorin 6A, TSK, Cytokeratin-8, CHST3, Mcl-1, DPPIV, SREC-II, Norrin, JAM-C, Bcl-10, Wnt-4, LSECtin, Kell, TNF RI, PTP1B, htPAPP-A, IDO, PDGF-CC, Galanin, Activin A, TLR2, SCCA2, FABP1, eNOS, SHP-1, ICOS, ClqTNF9, MMP-1, TC-PTP, IL-24, gp130, C-myc, LILRB4, BMP-2, MIA, CD34, CD63, CD9, CD81, IFNab R2, Glypican 2, MSP R, DSCAM, Matriptase, KIR2DL3, CD30, Siglec-10, CLEC-1, TPP1, Ubiquitin+1, ANGPTL4, TWEAK R, Nidogen-1, CD2, Kallikrein 1, TSLP R, LAMP1, TROY, VCAM-1, Siglec-11, S100A1, PAR1, Thyroid Peroxidase, Aminopeptidase P2, IL-1 RI, ADAMS, OSM R beta, Thrombospondin-2, SMPD1, B2M, MFRP, LRP-6, ST3GAL1, NCAM-1 (CD56), Granzyme B, Adiponectin, IL-22BP, TPST2, PD-ECGF, LH, LEDGF, Cyr61, ULBP-3, IFNb, THSD1, FGF-23, LAMA4, Adipsin, AIF, SorCS2, SULT2A1, CD39L2, Insulin R, HIF-1 alpha, OX40 Ligand, Pax3, UCH-L3, cMASP3, Langerin, Desmin, SOX9, ST6GAL1, MEP1B, CD99-L2, Plexin A4, Semaphorin 4D, ROBO2, PDX-1, APRIL, Neurturin, Kremen-2, EMMPRIN, Activin RIB, Neuroligin 2, Epiregulin, CASA, MMP-12, GALNT2, CEACAM-5, VEGF R1, DSPG3, SorCS1, Matrilin-2, sFRP-3, p53, EphB3, NCK1, Semaphorin 7A, NKp80, Prolactin, Cystatin B, Sirtuin 1, FGF-16, FGF R5, NQO-1, Semaphorin 6D, FGF-3, GATA-4, VAP-A, CHST2, Pappalysin-2, Syndecan-3, Jagged 1, AKR1C4, Olfactomedin-2, Osteoadherin, NKp44, Thyroglobulin, IL-21R, Chemerin, EphA1, CD48, MICB, FGF-5, TRANCE, CES2, ULBP-1, Integrin alpha 5, VAMP-2, FLRG, Ret Midkine, CD73, TRACP, proGRP, Granzyme H, PRX2, p2′7, Siglec-6, Dectin-1, CD51, Notch-1, Calreticulin, DR3, DCTN1, CDC25B, Osteoactivin, ACE, CA125, HAO-1, PSMA1, FCRLB, BMP-9, CRIM1, LIF, SPINK1, EphB6, RGM-B, HS3ST1, ROR1, CMG-2, 4-1BB Ligand, LiCAM-2, p63, Cathepsin V, Testican 2, Glypican 5, CD6, Siglec-2, Legumain, PRELP, CES1, TAZ, NSE, TECK, HTRA2, HIF-1 beta, TAFA1, Podocalyxin, RalA, CRELD2, GRAP2, SP-D, BID, GFR alpha-2, Notch-3, VEGF R3, DLL4, TGFb2, LIGHT, XIAP, ST8SIA1, Cathepsin L, 6Ckine, MIS RII, Kallikrein 5, TGM3, FCAR, Contactin-2, CD83, IL-1 R3, SALM4, GBA3, ROBO4, OSCAR, VEGF, IGSF3, Biglycan, Neudesin, ILT4, uPAR, Axl, WIF-1, IL-7 R alpha, GPR56, CEACAM-3, MCEMP1, FABP2, Plexin B3, MEPE, Activin RIIA, ANG-2, Cochlin, Presenilin 1, NPTXR, SLAM, COMT, SPHK1, RBP4, Nectin-1, GUSB, Nidogen-2, IL-17F, SR-AI, TAFA2, N-Cadherin, IL-17B, IL-17 RC, MIP-3b, Cystatin C, Cystatin D, AMSH, FcERI, CLEC10A, HGF R, ANG-1, Prolactin R, FGF-20, CD28, Nogo-A, HSD17B1, IL-19, Enteropeptidase, Cathepsin E, TSLP, TCN2, GDF-15, Epimorphin, GRKS, PD-1, Serpin A4, ADAM23, NOV, Galectin-2, Neurexin 3 beta, TLR3, Sirtuin 2, Numb, IL-28 R alpha, IL-33, Lin28, FCRL1, KLF4, NKp30, Lymphotactin, Cystatin SN, JAM-A, Calreticulin-2, ErbB4, BMP-8, IL-27 Ra, Fas, IL-4 Ra, Kallikrein 14, Matrilin-3, Olig2, Kallikrein 12, CA13, IL-9, Nectin-3, MPIF-1, Cystatin S, ADA, IL-2 Rb, GFR alpha-1, Smad4, ICAM-1, MEF2C, TREM-1, L-Selectin, Hepsin, CD42b, MCSF, RANK, CHST4, CA8, FCRL3, ASAH2, CF XIV, PYY, HGF, I-TAC, Semaphorin 4C, SorCS3, Tie-1, IL-31 RA, Arginase 1, POGLUT1, IL-1ra, Podoplanin, TIM-3, CREG, CD300f, uPA, EphA2, LRRTM4, LIMPII, Tenascin R, CPE, PECAM-1, DNAM-1, DKK-1, OPG, CPB1, TSH, MMP-2, Siglec-9, ICAM-3, Cystatin SA, Galectin-4, Pepsinogen IL, Desmoglein-3, Nectin-4, SCF, Serpin A5, PTH, FGF-19, MSP, IL-28A, FGF-12, METAP2, ASAHL, EDIL3, NTAL, EGF R, TAFAS, Galectin-9, vWF-A2, TACE, Activin RIM, Cathepsin S, LDL R, BMPR-IA, OX40, IL-13 R2, B7-H4, MMP-13, ANGPTL7, TRAIL R4, IGSF4B, Sirtuin 5, PEAR1, SH2D1A, Cerberus 1, GDF-11, Nrf2, TROP-2, NUDTS, ROR2, EphB4, Glypican 1, LAP(TGFb1), Gash, Contactin-1, IL-27, UNC5H4, ICAM-2, MBL, HS3ST3B1, RCOR1, IL-10 Rb, XEDAR, IL-22, PILR-alpha, NRG1-131, FABP4, RGM-A, RELT, TrkC, CSa, SREC-I, Nestin, TPO, ErbB3, Kirre13, FLRT1, Galectin-3, CXCL16, JAM-B, DR6, Nogo Receptor, TLR4, VEGF R2, Tie-2, IL-15 R, Caspr2, LTbR, LAMP, ALCAM, GLP-1, NG2, IL-22 R alpha 1, AMIGO2, HCC-1, TFPI-2, ULBP-2, Desmoglein 2, Aggrecan, Syntaxin 4, VAMP-1, Nectin-2, FGF-21, Flt-3, GFAP, TIM-1, Inhibin A, Cadherin-4, P1GF-2, Neurogranin, HE4, IL-23 R, Galectin-7, GALNT3, GITR L, CD14, R-Spondin 2, CK19, Cardiotrophin-1, TREML1, HAPLN1, CD27, ANG-4, Siglec-7, CD155, VEGF-C, TNF RII, PGRP-S, SDF-1a, PDGF-AB, GPVI, CD40, SCF R, Thrombospondin-5, IL-1 RII, Neuropilin-2, Cadherin-13, E-Selectin, GITR, WISP-1, Renin, AgRP, MDL-1, ROBO3, RANTES, Endocan, Granulysin, hCGb, Mesothelin, TLR1, TRAIL, MOG, DDR1, NGF R, TRAIL R3, Trypsin 3, ARSB, LIF R alpha, BAFF R, CD157, Granzyme A, 2B4, ESAM, IL-1 R4, CXCL14, IL-31, SIRP alpha, Uromodulin, CTRC, CEACAM-1, TARC, MIP-3a, SDF-1b, NKp46, MCP-3, IL-32 alpha, TGFb3 FOLR2, CD58, IL-23, CD36, TNFb, Shh-N, Ficolin-1, Reg4, ILT2, Mer, TREM-2, Flt-3L, CDS, IL-6, CD229, Insulin, Syntaxin 6, GRO, Bcl-w, Lipocalin-2, PDGF-AA, IL-2 Ra, Angiogenin, LYVE-1, CD4, RAGE, CDNF, Brevican, NAP-2, PU.1, EDAR, ADAMTS13, Kynureninase, PTH1R, IFN-gamma R1, CrkL, B7-1, PARC, Draxin, VE-Cadherin, Procalcitonin, SOX15, Kallikrein 11, BCMA, Dectin-2, EpCAM, HCC-4, TGFa, IP-10, BLAME, CILP-1, PIGF, LOX-1, MCP-2, Resistin, HVEM, ENPP-7, Syndecan-4, IL-2 Rg, MICA, Dopa Decarboxylase, NPDC-1, MCP-4, EG-VEGF, Glycoprotein V, Semaphorin 4G, IL-12p40, PSA-total, IL-15, MAP1D, C1q, TNF4, Dtk, Endoglin, ENA-78, Reg3A, MIP-1b, FGF-17, IL-6R, IL-8, Galectin-8, CA4, Cystatin E M, FUT8, B7-H3, GCP-2, CD40L, MDC, 4-1BB, HO-1, SOST, S100A13, Kallikrein 7, or IL-13. In some embodiments, the extracellular vesicles comprise one or more of the following nucleic acids: hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-1′7-5p, hsa-miR-181a-5p, hsa-miR-191-5p, hsa-miR-193a-5p, hsa-miR-193b-3p, hsa-miR-19′7-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, or hsa-miR-99b-5p. In some embodiments, the composition is produced by: (a) culturing bone marrow-derived MSCs under the following conditions to produce an MSC conditioned media: (i) oxygen tension below 5%; and (ii) culture media having a pH below 7; (b) harvesting the MSC conditioned media; and (c) formulating the MSC conditioned media to produce the therapeutic MSC secretome composition, wherein the therapeutic MSC secretome composition comprises proteins and extracellular vesicles produced by the bone marrow-derived MSCs in step (a). In some embodiments, the culture media is serum-free. In some embodiments, the culture media has a glucose concentration below 4.5 g/L. In some embodiments, the subject is human. In some embodiments, the bone-marrow derived MSCs are derived from human bone marrow. In some embodiments, administering comprises intravenous administration. In some embodiments, the dosage of the therapeutic MSC secretome composition administered to the subject is a cell-equivalent dosage of 0.7 to 7 million cells/kg. In some embodiments, the therapeutic MSC secretome composition comprises 4×10¹⁰ to 10×10¹⁰ cells/mL. In some embodiments, the therapeutic MSC secretome composition comprises 5×10¹¹ to 1.5×10¹² extracellular vesicles. In some embodiments, the composition is administered monthly for two or more months, or once every 1, 2, or 3 or more months. In some embodiments, the composition is administered twice. In some embodiments, the composition is administered 4 days apart.

An aspect of the present disclosure is a method of making a composition comprising a therapeutic mesenchymal stem cell (MSC) secretome composition for treating complex regional pain syndrome (CRPS) in a subject in need thereof, the method comprising: (a) culturing bone marrow-derived MSCs under the following conditions to produce an MSC conditioned media: (i) oxygen tension below 5%; and (ii) culture media having a pH below 7; (b) harvesting the MSC conditioned media; and (c) formulating the MSC conditioned media to produce the therapeutic MSC secretome composition, wherein the therapeutic MSC secretome composition comprises proteins and extracellular vesicles produced by the bone marrow-derived MSCs in step (a). In some embodiments, the culture media is serum-free. In some embodiments, the culture media has a glucose concentration below 4.5 g/L. In some embodiments, at least 80% of the extracellular vesicles in the therapeutic MSC secretome composition are CD63⁺ CD9⁻ CD81⁻. In some embodiments, the bone-marrow derived MSCs are derived from human bone marrow. In some embodiments, administering comprises intravenous administration. In some embodiments, the therapeutic MSC secretome composition further comprises one or more of the following proteins: Ferritin, NUP85, LAMP2, GPR115, Serpin F1, OPN, PAI-1, DAPP1, Cathepsin B, Semaphorin 6C, PDGF R alpha, Sortilin, Serpin B6, Dkk-3, Thrombomodulin, PF4, MIF, Periostin, Furin, TIMP-1, Decorin, PCK1, CD99, CD63, CD9, CD81, Transferrin, DcR3, Lumican, TIMP-2, SLITRK5, FAP, Artemin, DPPII, cIAP-1, Pentraxin 3, Visfatin, Neprilysin, Albumin, Galectin-1, UNC5H3, IL-20 R beta, SREC-IL, JAM-C, TNF RI, htPAPP-A, eNOS, MSP R, TPP1, LAMP1, B2M, NCAM-1, HIF-1 alpha, ST6GAL1, CD99-L2, Plexin A4, EMMPRIN, p53, Semaphorin 7A, NKp80, Cystatin B, Osteoadherin, Midkine, Calreticulin, Osteoactivin, Legumain, TAZ, Cathepsin L, RBP4, Serpin A4, JAM-A, MCSF, LIMPII, OPG, IL-22, Galectin-3, MOG, Trypsin 3, SIRP alpha, and Syndecan-4, and at least one protein selected from the following: Ferritin, IGFBP-4 IL-1 R6 GSTM1, NUP85, LAMP2, MeprinA, IL-1 F10, bIG-H3, GPR115, TGFb1, Ephrin-A4, CD109, Serpin F1, IGFBP-6, HS3ST4, Aminopeptidase LRAP, OPN, PAI-1, DAPP1, GDF-9, Cathepsin B, IGFBP-2, Semaphorin 6C, IGF-2, PDGF R alpha, Sortilin, Serpin B6, Dkk-3, CNTF, TSP-1, GM-CSF Ra, Thrombomodulin, Endoglycan, IGFBP-3, RGM-C, PF4, MIF, TGM4, Periostin, Furin, TIMP-1, PAPP-A, Decorin, PCK1, Arylsulfatase A, CD99, CA2, PRDX4, Transferrin, DcR3, GP73, LAIR2, ULBP-4, Lumican, TIMP-2, TFPI, SOX2, SLITRK5, FAP, Spinesin, ENPP-2, CD97, CTACK, Integrin alpha 1, EXTL3, IL-18 BPa, PD-L2, PSMA, IL-20 Ra, Glyoxalase II, Trypsin 1, IGF-2R, ADAMTSL-1, Erythropoietin, Plexin D1, DNMT3A, BCL-2, CL-P1, Ephrin-B3, FABP6, CHI3L1, FCRLS, TFF3, Artemin, DPPII, cIAP-1, PDGF Rb, Pentraxin 3, Angiotensinogen, Follistatin, CF VII, Persephin, TRAIL R1, THAP11, CD200, CLEC-2, AMIGO, IGFBP-5, PON1, SOX7, GALNT10, Visfatin, Progranulin, PCSK2, GKN1, IL-18, Neprilysin, Stabilin-2, IL-17 RD, Albumin, Follistatin-like 1, MMP-10, FKBP51, LRRC4, Pref-1, Galectin-1, Troponin C, UNC5H3, FLRT2, CD314, Semaphorin 6B, Netrin-4, CD27 Ligand, IL-20 R beta, Semaphorin 6A, TSK, Cytokeratin-8, CHST3, Mcl-1, DPPIV, SREC-IL, Norrin, JAM-C, Bcl-10, Wnt-4, LSECtin, Kell, TNF RI, PTP1B, htPAPP-A, IDO, PDGF-CC, Galanin, Activin A, TLR2, SCCA2, FABP1, eNOS, SHP-1, ICOS, ClqTNF9, MMP-1, TC-PTP, IL-24, gp130, C-myc, LILRB4, BMP-2, MIA, CD34, CD63, CD9, CD81, IFNab R2, Glypican 2, MSP R, DSCAM, Matriptase, KIR2DL3, CD30, Siglec-10, CLEC-1, TPP1, Ubiquitin+1, ANGPTL4, TWEAK R, Nidogen-1, CD2, Kallikrein 1, TSLP R, LAMP1, TROY, VCAM-1, Siglec-11, S100A1, PAR1, Thyroid Peroxidase, Aminopeptidase P2, IL-1 RI, ADAMS, OSM R beta, Thrombospondin-2, SMPD1, B2M, MFRP, LRP-6, ST3GAL1, NCAM-1 (CD56), Granzyme B, Adiponectin, IL-22BP, TPST2, PD-ECGF, LH, LEDGF, Cyr61, ULBP-3, IFNb, THSD1, FGF-23, LAMA4, Adipsin, AIF, SorCS2, SULT2A1, CD39L2, Insulin R, HIF-1 alpha, OX40 Ligand, Pax3, UCH-L3, cMASP3, Langerin, Desmin, SOX9, ST6GAL1, MEP1B, CD99-L2, Plexin A4, Semaphorin 4D, ROBO2, PDX-1, APRIL, Neurturin, Kremen-2, EMMPRIN, Activin RIB, Neuroligin 2, Epiregulin, CASA, MMP-12, GALNT2, CEACAM-5, VEGF R1, DSPG3, SorCS1, Matrilin-2, sFRP-3, p53, EphB3, NCK1, Semaphorin 7A, NKp80, Prolactin, Cystatin B, Sirtuin 1, FGF-16, FGF R5, NQO-1, Semaphorin 6D, FGF-3, GATA-4, VAP-A, CHST2, Pappalysin-2, Syndecan-3, Jagged 1, AKR1C4, Olfactomedin-2, Osteoadherin, NKp44, Thyroglobulin, IL-21R, Chemerin, EphA1, CD48, MICB, FGF-5, TRANCE, CES2, ULBP-1, Integrin alpha 5, VAMP-2, FLRG, Ret Midkine, CD73, TRACP, proGRP, Granzyme H, PRX2, p2′7, Siglec-6, Dectin-1, CD51, Notch-1, Calreticulin, DR3, DCTN1, CDC25B, Osteoactivin, ACE, CA125, HAO-1, PSMA1, FCRLB, BMP-9, CRIM1, LIF, SPINK1, EphB6, RGM-B, HS3ST1, ROR1, CMG-2, 4-1BB Ligand, L1CAM-2, p63, Cathepsin V, Testican 2, Glypican 5, CD6, Siglec-2, Legumain, PRELP, CES1, TAZ, NSE, TECK, HTRA2, HIF-1 beta, TAFA1, Podocalyxin, RalA, CRELD2, GRAP2, SP-D, BID, GFR alpha-2, Notch-3, VEGF R3, DLL4, TGFb2, LIGHT, XIAP, ST8SIA1, Cathepsin L, 6Ckine, MIS RII, Kallikrein 5, TGM3, FCAR, Contactin-2, CD83, IL-1 R3, SALM4, GBA3, ROBO4, OSCAR, VEGF, IGSF3, Biglycan, Neudesin, ILT4, uPAR, Axl, WIF-1, IL-7 R alpha, GPR56, CEACAM-3, MCEMP1, FABP2, Plexin B3, MEPE, Activin RIIA, ANG-2, Cochlin, Presenilin 1, NPTXR, SLAM, COMT, SPHK1, RBP4, Nectin-1, GUSB, Nidogen-2, IL-17F, SR-AI, TAFA2, N-Cadherin, IL-17B, IL-17 RC, MIP-3b, Cystatin C, Cystatin D, AMSH, FcERI, CLEC10A, HGF R, ANG-1, Prolactin R, FGF-20, CD28, Nogo-A, HSD17B1, IL-19, Enteropeptidase, Cathepsin E, TSLP, TCN2, GDF-15, Epimorphin, GRKS, PD-1, Serpin A4, ADAM23, NOV, Galectin-2, Neurexin 3 beta, TLR3, Sirtuin 2, Numb, IL-28 R alpha, IL-33, Lin28, FCRL1, KLF4, NKp30, Lymphotactin, Cystatin SN, JAM-A, Calreticulin-2, ErbB4, BMP-8, IL-27 Ra, Fas, IL-4 Ra, Kallikrein 14, Matrilin-3, Olig2, Kallikrein 12, CA13, IL-9, Nectin-3, MPIF-1, Cystatin S, ADA, IL-2 Rb, GFR alpha-1, Smad4, ICAM-1, MEF2C, TREM-1, L-Selectin, Hepsin, CD42b, MCSF, RANK, CHST4, CA8, FCRL3, ASAH2, CF XIV, PYY, HGF, I-TAC, Semaphorin 4C, SorCS3, Tie-1, IL-31 RA, Arginase 1, POGLUT1, IL-Ira, Podoplanin, TIM-3, CREG, CD300f, uPA, EphA2, LRRTM4, LIMPII, Tenascin R, CPE, PECAM-1, DNAM-1, DKK-1, OPG, CPB1, TSH, MMP-2, Siglec-9, ICAM-3, Cystatin SA, Galectin-4, Pepsinogen IL, Desmoglein-3, Nectin-4, SCF, Serpin A5, PTH, FGF-19, MSP, IL-28A, FGF-12, METAP2, ASAHL, EDIL3, NTAL, EGF R, TAFAS, Galectin-9, vWF-A2, TACE, Activin RIM, Cathepsin S, LDL R, BMPR-IA, OX40, IL-13 R2, B7-H4, MMP-13, ANGPTL7, TRAIL R4, IGSF4B, Sirtuin 5, PEAR1, SH2D1A, Cerberus 1, GDF-11, Nrf2, TROP-2, NUDTS, ROR2, EphB4, Glypican 1, LAP(TGFb1), Gash, Contactin-1, IL-27, UNC5H4, ICAM-2, MBL, HS3ST3B1, RCOR1, IL-10 Rb, XEDAR, IL-22, PILR-alpha, NRG1-131, FABP4, RGM-A, RELT, TrkC, CSa, SREC-I, Nestin, TPO, ErbB3, Kirre13, FLRT1, Galectin-3, CXCL16, JAM-B, DR6, Nogo Receptor, TLR4, VEGF R2, Tie-2, IL-15 R, Caspr2, LTbR, LAMP, ALCAM, GLP-1, NG2, IL-22 R alpha 1, AMIGO2, HCC-1, TFPI-2, ULBP-2, Desmoglein 2, Aggrecan, Syntaxin 4, VAMP-1, Nectin-2, FGF-21, Flt-3, GFAP, TIM-1, Inhibin A, Cadherin-4, PIGF-2, Neurogranin, HE4, IL-23 R, Galectin-7, GALNT3, GITR L, CD14, R-Spondin 2, CK19, Cardiotrophin-1, TREML1, HAPLN1, CD27, ANG-4, Siglec-7, CD155, VEGF-C, TNF RII, PGRP-S, SDF-1a, PDGF-AB, GPVI, CD40, SCF R, Thrombospondin-5, IL-1 RII, Neuropilin-2, Cadherin-13, E-Selectin, GITR, WISP-1, Renin, AgRP, MDL-1, ROBO3, RANTES, Endocan, Granulysin, hCGb, Mesothelin, TLR1, TRAIL, MOG, DDR1, NGF R, TRAIL R3, Trypsin 3, ARSB, LIF R alpha, BAFF R, CD157, Granzyme A, 2B4, ESAM, IL-1 R4, CXCL14, IL-31, SIRP alpha, Uromodulin, CTRC, CEACAM-1, TARC, MIP-3a, SDF-1b, NKp46, MCP-3, IL-32 alpha, TGFb3 FOLR2, CD58, IL-23, CD36, TNFb, Shh-N, Ficolin-1, Reg4, ILT2, Mer, TREM-2, Flt-3L, CDS, IL-6, CD229, Insulin, Syntaxin 6, GRO, Bcl-w, Lipocalin-2, PDGF-AA, IL-2 Ra, Angiogenin, LYVE-1, CD4, RAGE, CDNF, Brevican, NAP-2, PU.1, EDAR, ADAMTS13, Kynureninase, PTH1R, IFN-gamma R1, CrkL, B7-1, PARC, Draxin, VE-Cadherin, Procalcitonin, SOX15, Kallikrein 11, BCMA, Dectin-2, EpCAM, HCC-4, TGFa, IP-10, BLAME, CILP-1, PIGF, LOX-1, MCP-2, Resistin, HVEM, ENPP-7, Syndecan-4, IL-2 Rg, MICA, Dopa Decarboxylase, NPDC-1, MCP-4, EG-VEGF, Glycoprotein V, Semaphorin 4G, IL-12p40, PSA-total, IL-15, MAP1D, C1q, TNF4, Dtk, Endoglin, ENA-78, Reg3A, MIP-1b, FGF-17, IL-6R, IL-8, Galectin-8, CA4, Cystatin E M, FUT8, B7-H3, GCP-2, CD40L, MDC, 4-1BB, HO-1, SOST, S100A13, Kallikrein 7, or IL-13. In some embodiments, the extracellular vesicles comprise one or more of the following nucleic acids: hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-1′7-5p, hsa-miR-181a-5p, hsa-miR-191-5p, hsa-miR-193a-5p, hsa-miR-193b-3p, hsa-miR-19′7-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, or hsa-miR-99b-5p.

An aspect of the present disclosure is the use of the composition produced by the method of any one of the present disclosure for treating complex regional pain syndrome (CRPS) in a subject in need thereof. In some embodiments, the subject is human. In some embodiments, the subject has a decrease of at least 50% in a Visual Analog Scale of Pain (VAS) score after administration compared to before administration. In some embodiments, the subject has a minimum VAS score of greater than 6 in an affected extremity prior to administration. In some embodiments, the subject has a decrease in a Brief Pain Inventory (BPI) score after administration compared to before administration. In some embodiments, the subject has a decrease in a BPI score of at least 10%, at least 20%, at least 30%, or greater than 30% after administration compared to before administration. In some embodiments, the subject has a decrease in a BPI pain interference score of at least 10%, at least 20%, at least 30%, or greater than 30% after administration compared to before administration. In some embodiments, the subject has an increase in a range of motion assessment score of at least 50%, at least 60%, at least 70%, at least 75%, at least 80%, at least 85%, at least 95%, or at least 100% after administration compared to before administration. In some embodiments, the subject has an increase in a Jamar dynamometer test assessment score after administration as compared to before administration. In some embodiments, the increase in the Jamar dynamometer test assessment score is in an upper extremity. In some embodiments, the subject has an equalization of grip strength after administration. In some embodiments, the subject has an increase in grip strength of at least 5 kg, at least 10 kg, at least 15 kg, at or least 20 kg after administration compared to before administration. In some embodiments, the subject has an increase in grip strength of at least 50%, at least 75%, at least 100%, at least 125%, at least 150%, at least 175%, at least 200%, or at least 210% after administration compared to before administration. In some embodiments, the subject has an increase in grip strength in an affected limb. In some embodiments, the subject has an increase in grip strength in a control limb. In some embodiments, the subject has an improvement after administration compared to before administration in one or more of: limb range of motion, joint range of motion, sudomotor changes, edema, allodynia, and hypesthesia. In some embodiments, the subject has an improvement in an Short Form 36 Health Survey Questionnaire (SF-36) Questionnaire score after administration compared to before administration. In some embodiments, the improvement in the SF-36 Questionnaire score comprises one or more of: an improvement in pain, an improvement in energy levels, an improvement in social functioning, an improvement in physical functioning, a positive trend in emotional wellbeing, and an improvement in general health. In some embodiments, the subject has chronic intractable pain of one or more upper or lower extremities. In some embodiments, the subject does not experience escalating or changing pain in the 30 days preceding administration. In some embodiments, the subject experiences decreased pain flare-ups, decreased swelling, and/or decreased burning sensations in an affected limb after administration compared to before administration. In some embodiments, the administration improves hair and/or nail growth in an affected limb of the subject. In some embodiments, the dosage of the therapeutic MSC secretome composition administered to the subject is a cell-equivalent dosage of 0.7 to 7 million cells/kg. In some embodiments, the therapeutic MSC secretome composition comprises 4×10¹⁰ to 10×10¹⁰ cells/mL. In some embodiments, the therapeutic MSC secretome composition comprises 5×10¹¹ to 1.5×10¹² extracellular vesicles. In some embodiments, the composition is administered monthly for two or more months, or once every 1, 2, or 3 or more months. In some embodiments, the composition is administered twice. In some embodiments, the composition is administered 4 days apart.

An aspect of the present disclosure is the use of a composition comprising a therapeutic mesenchymal stem cell (MSC) secretome composition comprising extracellular vesicles in treating complex regional pain syndrome (CRPS) in a subject in need thereof, wherein at least 80% of the extracellular vesicles in the therapeutic MSC secretome composition are CD63+ CD9− CD81−. In some embodiments, the composition is intravenously administered to the subject. In some embodiments, the subject has a decrease of at least 50% in a Visual Analog Scale of Pain (VAS) score after administration of the therapeutic MSC secretome composition compared to before administration of the therapeutic MSC secretome composition. In some embodiments, the subject has a minimum VAS score of greater than 6 in an affected extremity prior to administration of the therapeutic MSC secretome composition. In some embodiments, the subject has a decrease in a Brief Pain Inventory (BPI) score after administration of the therapeutic MSC secretome composition compared to before administration of the therapeutic MSC secretome composition.

An aspect of the present disclosure is the use of a composition comprising a therapeutic mesenchymal stem cell (MSC) secretome composition comprising extracellular vesicles in treating complex regional pain syndrome (CRPS) in a subject in need thereof, wherein the subject has a decrease of at least 50% in a Visual Analog Scale of Pain (VAS) score after administration of the therapeutic MSC secretome composition. compared to before administration of the therapeutic MSC secretome composition. In some embodiments, the subject has a minimum VAS score of greater than 6 in an affected extremity prior to administration of the therapeutic MSC secretome composition. In some embodiments, the subject has a decrease in a Brief Pain Inventory (BPI) score after administration of the therapeutic MSC secretome composition compared to before administration of the therapeutic MSC secretome composition. In some embodiments, at least 80% of the extracellular vesicles in the therapeutic MSC secretome composition are CD63⁺ CD9⁻ CD81⁻. In some embodiments, the therapeutic MSC secretome composition further comprises one or more of the following proteins: Ferritin, NUP85, LAMP2, GPR115, Serpin F1, OPN, PAI-1, DAPP1, Cathepsin B, Semaphorin 6C, PDGF R alpha, Sortilin, Serpin B6, Dkk-3, Thrombomodulin, PF4, MIF, Periostin, Furin, TIMP-1, Decorin, PCK1, CD99, CD63, CD9, CD81, Transferrin, DcR3, Lumican, TIMP-2, SLITRK5, FAP, Artemin, DPPII, cIAP-1, Pentraxin 3, Visfatin, Neprilysin, Albumin, Galectin-1, UNC5H3, IL-20 R beta, SREC-II, JAM-C, TNF RI, htPAPP-A, eNOS, MSP R, TPP1, LAMP1, B2M, NCAM-1, HIF-1 alpha, ST6GAL1, CD99-L2, Plexin A4, EMMPRIN, p53, Semaphorin 7A, NKp80, Cystatin B, Osteoadherin, Midkine, Calreticulin, Osteoactivin, Legumain, TAZ, Cathepsin L, RBP4, Serpin A4, JAM-A, MCSF, LIMPII, OPG, IL-22, Galectin-3, MOG, Trypsin 3, SIRP alpha, and Syndecan-4, and at least one protein selected from the following: Ferritin, IGFBP-4 IL-1 R6 GSTM1, NUP85, LAMP2, MeprinA, IL-1 F10, bIG-H3, GPR115, TGFb1, Ephrin-A4, CD109, Serpin F1, IGFBP-6, HS3ST4, Aminopeptidase LRAP, OPN, PAI-1, DAPP1, GDF-9, Cathepsin B, IGFBP-2, Semaphorin 6C, IGF-2, PDGF R alpha, Sortilin, Serpin B6, Dkk-3, CNTF, TSP-1, GM-CSF Ra, Thrombomodulin, Endoglycan, IGFBP-3, RGM-C, PF4, MIF, TGM4, Periostin, Furin, TIMP-1, PAPP-A, Decorin, PCK1, Arylsulfatase A, CD99, CA2, PRDX4, Transferrin, DcR3, GP73, LAIR2, ULBP-4, Lumican, TIMP-2, TFPI, SOX2, SLITRK5, FAP, Spinesin, ENPP-2, CD97, CTACK, Integrin alpha 1, EXTL3, IL-18 BPa, PD-L2, PSMA, IL-20 Ra, Glyoxalase II, Trypsin 1, IGF-2R, ADAMTSL-1, Erythropoietin, Plexin D1, DNMT3A, BCL-2, CL-P1, Ephrin-B3, FABP6, CHI3L1, FCRLS, TFF3, Artemin, DPPII, cIAP-1, PDGF Rb, Pentraxin 3, Angiotensinogen, Follistatin, CF VII, Persephin, TRAIL R1, THAP11, CD200, CLEC-2, AMIGO, IGFBP-5, PON1, SOX7, GALNT10, Visfatin, Progranulin, PCSK2, GKN1, IL-18, Neprilysin, Stabilin-2, IL-17 RD, Albumin, Follistatin-like 1, MMP-10, FKBP51, LRRC4, Pref-1, Galectin-1, Troponin C, UNC5H3, FLRT2, CD314, Semaphorin 6B, Netrin-4, CD27 Ligand, IL-20 R beta, Semaphorin 6A, TSK, Cytokeratin-8, CHST3, Mcl-1, DPPIV, SREC-II, Norrin, JAM-C, Bcl-10, Wnt-4, LSECtin, Kell, TNF RI, PTP1B, htPAPP-A, IDO, PDGF-CC, Galanin, Activin A, TLR2, SCCA2, FABP1, eNOS, SHP-1, ICOS, ClqTNF9, MMP-1, TC-PTP, IL-24, gp130, C-myc, LILRB4, BMP-2, MIA, CD34, CD63, CD9, CD81, IFNab R2, Glypican 2, MSP R, DSCAM, Matriptase, KIR2DL3, CD30, Siglec-10, CLEC-1, TPP1, Ubiquitin+1, ANGPTL4, TWEAK R, Nidogen-1, CD2, Kallikrein 1, TSLP R, LAMP1, TROY, VCAM-1, Siglec-11, S100A1, PAR1, Thyroid Peroxidase, Aminopeptidase P2, IL-1 RI, ADAMS, OSM R beta, Thrombospondin-2, SMPD1, B2M, MFRP, LRP-6, ST3GAL1, NCAM-1 (CD56), Granzyme B, Adiponectin, IL-22BP, TPST2, PD-ECGF, LH, LEDGF, Cyr61, ULBP-3, IFNb, THSD1, FGF-23, LAMA4, Adipsin, AIF, SorCS2, SULT2A1, CD39L2, Insulin R, HIF-1 alpha, OX40 Ligand, Pax3, UCH-L3, cMASP3, Langerin, Desmin, SOX9, ST6GAL1, MEP1B, CD99-L2, Plexin A4, Semaphorin 4D, ROBO2, PDX-1, APRIL, Neurturin, Kremen-2, EMMPRIN, Activin RIB, Neuroligin 2, Epiregulin, CASA, MMP-12, GALNT2, CEACAM-5, VEGF R1, DSPG3, SorCS1, Matrilin-2, sFRP-3, p53, EphB3, NCK1, Semaphorin 7A, NKp80, Prolactin, Cystatin B, Sirtuin 1, FGF-16, FGF R5, NQO-1, Semaphorin 6D, FGF-3, GATA-4, VAP-A, CHST2, Pappalysin-2, Syndecan-3, Jagged 1, AKR1C4, Olfactomedin-2, Osteoadherin, NKp44, Thyroglobulin, IL-21R, Chemerin, EphA1, CD48, MICB, FGF-5, TRANCE, CES2, ULBP-1, Integrin alpha 5, VAMP-2, FLRG, Ret Midkine, CD73, TRACP, proGRP, Granzyme H, PRX2, p2′7, Siglec-6, Dectin-1, CD51, Notch-1, Calreticulin, DR3, DCTN1, CDC25B, Osteoactivin, ACE, CA125, HAO-1, PSMA1, FCRLB, BMP-9, CRIM1, LIF, SPINK1, EphB6, RGM-B, HS3ST1, ROR1, CMG-2, 4-1BB Ligand, LiCAM-2, p63, Cathepsin V, Testican 2, Glypican 5, CD6, Siglec-2, Legumain, PRELP, CES1, TAZ, NSE, TECK, HTRA2, HIF-1 beta, TAFA1, Podocalyxin, RalA, CRELD2, GRAP2, SP-D, BID, GFR alpha-2, Notch-3, VEGF R3, DLL4, TGFb2, LIGHT, XIAP, ST8SIA1, Cathepsin L, 6Ckine, MIS RII, Kallikrein 5, TGM3, FCAR, Contactin-2, CD83, IL-1 R3, SALM4, GBA3, ROBO4, OSCAR, VEGF, IGSF3, Biglycan, Neudesin, ILT4, uPAR, Axl, WIF-1, IL-7 R alpha, GPR56, CEACAM-3, MCEMP1, FABP2, Plexin B3, MEPE, Activin RIIA, ANG-2, Cochlin, Presenilin 1, NPTXR, SLAM, COMT, SPHK1, RBP4, Nectin-1, GUSB, Nidogen-2, IL-17F, SR-AI, TAFA2, N-Cadherin, IL-17B, IL-17 RC, MIP-3b, Cystatin C, Cystatin D, AMSH, FcERI, CLEC10A, HGF R, ANG-1, Prolactin R, FGF-20, CD28, Nogo-A, HSD17B1, IL-19, Enteropeptidase, Cathepsin E, TSLP, TCN2, GDF-15, Epimorphin, GRKS, PD-1, Serpin A4, ADAM23, NOV, Galectin-2, Neurexin 3 beta, TLR3, Sirtuin 2, Numb, IL-28 R alpha, IL-33, Lin28, FCRL1, KLF4, NKp30, Lymphotactin, Cystatin SN, JAM-A, Calreticulin-2, ErbB4, BMP-8, IL-27 Ra, Fas, IL-4 Ra, Kallikrein 14, Matrilin-3, Olig2, Kallikrein 12, CA13, IL-9, Nectin-3, MPIF-1, Cystatin S, ADA, IL-2 Rb, GFR alpha-1, Smad4, ICAM-1, MEF2C, TREM-1, L-Selectin, Hepsin, CD42b, MCSF, RANK, CHST4, CA8, FCRL3, ASAH2, CF XIV, PYY, HGF, I-TAC, Semaphorin 4C, SorCS3, Tie-1, IL-31 RA, Arginase 1, POGLUT1, IL-Ira, Podoplanin, TIM-3, CREG, CD300f, uPA, EphA2, LRRTM4, LIMPII, Tenascin R, CPE, PECAM-1, DNAM-1, DKK-1, OPG, CPB1, TSH, MMP-2, Siglec-9, ICAM-3, Cystatin SA, Galectin-4, Pepsinogen II, Desmoglein-3, Nectin-4, SCF, Serpin A5, PTH, FGF-19, MSP, IL-28A, FGF-12, METAP2, ASAHL, EDIL3, NTAL, EGF R, TAFAS, Galectin-9, vWF-A2, TACE, Activin RIM, Cathepsin S, LDL R, BMPR-IA, OX40, IL-13 R2, B7-H4, MMP-13, ANGPTL7, TRAIL R4, IGSF4B, Sirtuin 5, PEAR1, SH2D1A, Cerberus 1, GDF-11, Nrf2, TROP-2, NUDTS, ROR2, EphB4, Glypican 1, LAP(TGFb1), Gash, Contactin-1, IL-27, UNC5H4, ICAM-2, MBL, HS3ST3B1, RCOR1, IL-10 Rb, XEDAR, IL-22, PILR-alpha, NRG1-131, FABP4, RGM-A, RELT, TrkC, CSa, SREC-I, Nestin, TPO, ErbB3, Kirre13, FLRT1, Galectin-3, CXCL16, JAM-B, DR6, Nogo Receptor, TLR4, VEGF R2, Tie-2, IL-15 R, Caspr2, LTbR, LAMP, ALCAM, GLP-1, NG2, IL-22 R alpha 1, AMIGO2, HCC-1, TFPI-2, ULBP-2, Desmoglein 2, Aggrecan, Syntaxin 4, VAMP-1, Nectin-2, FGF-21, Flt-3, GFAP, TIM-1, Inhibin A, Cadherin-4, P1GF-2, Neurogranin, HE4, IL-23 R, Galectin-7, GALNT3, GITR L, CD14, R-Spondin 2, CK19, Cardiotrophin-1, TREML1, HAPLN1, CD27, ANG-4, Siglec-7, CD155, VEGF-C, TNF RI, PGRP-S, SDF-1a, PDGF-AB, GPVI, CD40, SCF R, Thrombospondin-5, IL-1 RII, Neuropilin-2, Cadherin-13, E-Selectin, GITR, WISP-1, Renin, AgRP, MDL-1, ROBO3, RANTES, Endocan, Granulysin, hCGb, Mesothelin, TLR1, TRAIL, MOG, DDR1, NGF R, TRAIL R3, Trypsin 3, ARSB, LIF R alpha, BAFF R, CD157, Granzyme A, 2B4, ESAM, IL-1 R4, CXCL14, IL-31, SIRP alpha, Uromodulin, CTRC, CEACAM-1, TARC, MIP-3a, SDF-1b, NKp46, MCP-3, IL-32 alpha, TGFb3 FOLR2, CD58, IL-23, CD36, TNFb, Shh-N, Ficolin-1, Reg4, ILT2, Mer, TREM-2, Flt-3L, CDS, IL-6, CD229, Insulin, Syntaxin 6, GRO, Bcl-w, Lipocalin-2, PDGF-AA, IL-2 Ra, Angiogenin, LYVE-1, CD4, RAGE, CDNF, Brevican, NAP-2, PU.1, EDAR, ADAMTS13, Kynureninase, PTH1R, IFN-gamma R1, CrkL, B7-1, PARC, Draxin, VE-Cadherin, Procalcitonin, SOX15, Kallikrein 11, BCMA, Dectin-2, EpCAM, HCC-4, TGFa, IP-10, BLAME, CILP-1, PIGF, LOX-1, MCP-2, Resistin, HVEM, ENPP-7, Syndecan-4, IL-2 Rg, MICA, Dopa Decarboxylase, NPDC-1, MCP-4, EG-VEGF, Glycoprotein V, Semaphorin 4G, IL-12p40, PSA-total, IL-15, MAP1D, C1q, TNF4, Dtk, Endoglin, ENA-78, Reg3A, MIP-1b, FGF-17, IL-6R, IL-8, Galectin-8, CA4, Cystatin E M, FUT8, B7-H3, GCP-2, CD40L, MDC, 4-1BB, HO-1, SOST, S100A13, Kallikrein 7, or IL-13. In some embodiments, the extracellular vesicles comprise one or more of the following nucleic acids: hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-1′7-5p, hsa-miR-181a-5p, hsa-miR-191-5p, hsa-miR-193a-5p, hsa-miR-193b-3p, hsa-miR-19′7-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, or hsa-miR-99b-5p. In some embodiments, the composition is produced by: (a) culturing bone marrow-derived MSCs under the following conditions to produce an MSC conditioned media: (i) oxygen tension below 5%; and (ii) culture media having a pH below 7; (b) harvesting the MSC conditioned media; and (c) formulating the MSC conditioned media to produce the therapeutic MSC secretome composition, wherein the therapeutic MSC secretome composition comprises proteins and extracellular vesicles produced by the bone marrow-derived MSCs in step (a). In some embodiments, the culture media is serum-free. In some embodiments, the culture media has a glucose concentration below 4.5 g/L. In some embodiments, the subject has a decrease in a BPI score of at least 10%, at least 20%, at least 30%, or greater than 30% after administration of the therapeutic MSC secretome composition compared to before administration of the therapeutic MSC secretome composition. In some embodiments, the subject has a decrease in a BPI pain interference score of at least 10%, at least 20%, at least 30%, or greater than 30% after administration of the therapeutic MSC secretome composition compared to before administration of the therapeutic MSC secretome composition. In some embodiments, the subject has an increase in a range of motion assessment score of at least 50%, at least 60%, at least 70%, at least 75%, at least 80%, at least 85%, at least 95%, or at least 100% after administration of the therapeutic MSC secretome composition compared to before administration of the therapeutic MSC secretome composition. In some embodiments, the subject has an increase in a Jamar dynamometer test assessment score after administration of the therapeutic MSC secretome composition as compared to before administration of the therapeutic MSC secretome composition. In some embodiments, the increase in the Jamar dynamometer test assessment score is in an upper extremity. In some embodiments, the subject has an equalization of grip strength after administration of the therapeutic MSC secretome composition. In some embodiments, the subject has an increase in grip strength of at least 5 kg, at least 10 kg, at least 15 kg, at or least 20 kg after administration of the therapeutic MSC secretome composition compared to before administration of the therapeutic MSC secretome composition. In some embodiments, the subject has an increase in grip strength of at least 50%, at least 75%, at least 100%, at least 125%, at least 150%, at least 175%, at least 200%, or at least 210% after administration of the therapeutic MSC secretome composition compared to before administration of the therapeutic MSC secretome composition. In some embodiments, the subject has an increase in grip strength in an affected limb. In some embodiments, the subject has an increase in grip strength in a control limb. In some embodiments, the subject has an improvement after administration of the therapeutic MSC secretome composition compared to before administration of the therapeutic MSC secretome composition in one or more of: limb range of motion, joint range of motion, sudomotor changes, edema, allodynia, and hypesthesia. In some embodiments, the subject has an improvement in an Short Form 36 Health Survey Questionnaire (SF-36) Questionnaire score after administration of the therapeutic MSC secretome composition compared to before administration of the therapeutic MSC secretome composition. In some embodiments, the improvement in the SF-36 Questionnaire score comprises one or more of: an improvement in pain, an improvement in energy levels, an improvement in social functioning, an improvement in physical functioning, a positive trend in emotional wellbeing, and an improvement in general health. In some embodiments, the subject has chronic intractable pain of one or more upper or lower extremities. In some embodiments, the subject does not experience escalating or changing pain in the 30 days preceding administration of the therapeutic MSC secretome composition. In some embodiments, the subject experiences decreased pain flare-ups, decreased swelling, and/or decreased burning sensations in an affected limb after administration of the therapeutic MSC secretome composition compared to before administration of the therapeutic MSC secretome composition. In some embodiments, the administration of the therapeutic MSC secretome composition improves hair and/or nail growth in an affected limb of the subject.

### INCORPORATION BY REFERENCE

Each patent, publication, and non-patent literature cited in the application is hereby incorporated by reference in its entirety as if each was incorporated by reference individually. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows Box and Whisker plot of Mean Visual Analog Scale (VAS) pain assessment results over the 6-month study period. The box identifies the 25th and 75th percentiles with the interior line identifying the median value and the ‘+’ identifying the mean value. Upper and lower whisker lines denote maximal and minimal data values, respectively. Individual data points are displayed as single dots (•). (*p<0.05; ** p<0.01).

FIG. 2 shows plots showing Brief Pain Index (A, pain interference and B, pain severity) assessment results over the 6-month study period. Plots and p value markers are as in FIG. 1.

FIG. 3 shows plots showing SF-36 assessment results over the 6-month study period. Categories are A) Pain, B) Energy/Fatigue, C) Social Functioning, D) Physical Functioning, E) Limitations Physical, F) Limitations Emotional, G) Emotional Wellbeing and H) General Health. Plots and p value markers are as in FIG. 1.

FIG. 4 shows range of motion assessment results over the 6-month study period shown as a bar graph.

FIG. 5 shows Jamar dynamometer test assessment results over the 6-month study period quantified in a bar graph.

### DETAILED DESCRIPTION

### I. Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value, which may depend in part on how the value is measured or determined, e.g., the limitations of the measurement system. For example, “about” can mean within 1 or more than 1 standard deviation. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the present disclosure, subject to any specifically excluded limit in the stated range. Where the stated range includes one or both of the limits, ranges excluding either or both of those included limits are also included in the present disclosure, unless the context clearly dictates otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Certain specific details of this description are set forth in order to provide a thorough understanding of various embodiments. However, one skilled in the art will understand that the present disclosure may be practiced without these details. In other instances, well-known techniques or methods have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

Although methods and materials similar or equivalent to those described herein can be used in the practice or testing of the present disclosure, suitable methods, and materials are described below.

### II. Therapeutic Composition

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular MSC secretome (including, but not limited to a MSC exosome (with or without growth factors) referred to herein as an extracellular vesicle isolate product (EVIP)) is disclosed and discussed and a number of modifications that can be made to a number of molecules including the MSC secretome are discussed, specifically contemplated is each and every combination and permutation of MSC secretome and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

A primary trophic property of MSCs is the secretion of growth factors and exosomes to induce cell proliferation and angiogenesis. Exosomes express mitogenic proteins such as transforming growth factor-alpha (TGF-α), TGFβ, hepatocyte growth factor (HGF), epithelial growth factor (EGF), basic fibroblast growth factor (FGF-2) and insulin-like growth factor-1 (IGF-1). These increase fibroblast, epithelial and endothelial cell division. Vascular endothelial growth factor (VEGF), IGF-1, EGF and angiopoietin-1 are released to recruit endothelial lineage cells and initiate vascularization. MSCs assist via paracrine mechanisms and modulate the regenerative environment via anti-inflammatory and immunomodulatory mechanisms. In response to inflammatory molecules such as interleukin-1 (IL-1), IL-6, IL-2, IL-12, tumor necrosis factor-a (TNF-α) and interferon-gamma (INF-γ), MSCs secrete an array of growth factors and anti-inflammatory proteins with complex feedback mechanisms among the many types of immune cells. The key immunomodulatory cytokines include prostaglandin 2, TGF-131, HGF, SDF-1, nitrous oxide, indoleamine 2, 3-dioxygenase, IL-4, IL-10, IL-1 receptor antagonist and soluble tumor necrosis factor-a receptor. MSCs prevent proliferation and function of many inflammatory immune cells, including T-cells, natural killer cells, B-cells, monocytes, macrophages, and dendritic cells. Although MSCs across species are able to regulate T-cell activity, the mechanisms are not identical across mammalian species.

A characteristic of chronically inflamed environments is a persistent imbalance in the types of helper T-cells and macrophages. MSC exosomes indirectly promote the transition of TH1 to TH2 cells by reducing INF-γ and increasing IL-4 and IL-10. The restored TH1/TH2 balance has been shown to improve tissue regeneration in cartilage, muscle, and other soft tissue injuries, alleviate symptoms of autoimmune diseases, and have an anti-diabetic effect. Similarly, reduction in INF-γ and secretion of IL-4 promotes a shift in macrophages from M1 (proinflammatory, anti-angiogenic and tissue growth inhibition) to M2 (anti-inflammatory, pro-remodeling and tissue healing) type, an effect required for skeletal, muscular, and neural healing and regeneration.

Disclosed herein is a complex composition of secreted biomolecules (proteins, lipids, and ribonucleic acids) and/or extracellular vesicles comprising biomolecules, originating from mesenchymal lineage cells. In one aspect, disclosed herein are compositions comprising a therapeutically effective amount of a MSC secretome (such as, for example, including, but not limited to MSC growth factor, MSC exosome, MSC extracts and/or extracellular vesicle comprising compositions) and one or more biomolecules (such as, for example, a peptide, polypeptide, protein, siRNA, shRNA, and/or microRNA (miRNA)).

In some embodiments, the therapeutic composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, or 100 or more of the following proteins, in any combination, or all of the following proteins: Ferritin, NUP85, LAMP2, GPR115, Serpin F1, OPN, PAI-1, DAPP1, Cathepsin B, Semaphorin 6C, PDGF R alpha, Sortilin, Serpin B6, Dkk-3, Thrombomodulin, PF4, MIF, Periostin, Furin, TIMP-1, Decorin, PCK1, CD99, CD63, CD9, CD81, Transferrin, DcR3, Lumican, TIMP-2, SLITRK5, FAP, Artemin, DPPII, cIAP-1, Pentraxin 3, Visfatin, Neprilysin, Albumin, Galectin-1, UNC5H3, IL-20 R beta, SREC-II, JAM-C, TNF RI, htPAPP-A, eNOS, MSP R, TPP1, LAMP1, B2M, NCAM-1, HIF-1 alpha, ST6GAL1, CD99-L2, Plexin A4, EMMPRIN, p53, Semaphorin 7A, NKp80, Cystatin B, Osteoadherin, Midkine, Calreticulin, Osteoactivin, Legumain, TAZ, Cathepsin L, RBP4, Serpin A4, JAM-A, MCSF, LIMPII, OPG, IL-22, Galectin-3, MOG, Trypsin 3, SIRP alpha, and Syndecan-4, and at least one protein selected from the following: Ferritin, IGFBP-4 IL-1 R6 GSTM1, NUP85, LAMP2, MeprinA, IL-1 F10, bIG-H3, GPR115, TGFb1, Ephrin-A4, CD109, Serpin F1, IGFBP-6, HS3ST4, Aminopeptidase LRAP, OPN, PAI-1, DAPP1, GDF-9, Cathepsin B, IGFBP-2, Semaphorin 6C, IGF-2, PDGF R alpha, Sortilin, Serpin B6, Dkk-3, CNTF, TSP-1, GM-CSF Ra, Thrombomodulin, Endoglycan, IGFBP-3, RGM-C, PF4, MIF, TGM4, Periostin, Furin, TIMP-1, PAPP-A, Decorin, PCK1, Arylsulfatase A, CD99, CA2, PRDX4, Transferrin, DcR3, GP73, LAIR2, ULBP-4, Lumican, TIMP-2, TFPI, SOX2, SLITRK5, FAP, Spinesin, ENPP-2, CD97, CTACK, Integrin alpha 1, EXTL3, IL-18 BPa, PD-L2, PSMA, IL-20 Ra, Glyoxalase II, Trypsin 1, IGF-2R, ADAMTSL-1, Erythropoietin, Plexin D1, DNMT3A, BCL-2, CL-P1, Ephrin-B3, FABP6, CHI3L1, FCRLS, TFF3, Artemin, DPPII, cIAP-1, PDGF Rb, Pentraxin 3, Angiotensinogen, Follistatin, CF VII, Persephin, TRAIL R1, THAP11, CD200, CLEC-2, AMIGO, IGFBP-5, PON1, SOX7, GALNT10, Visfatin, Progranulin, PCSK2, GKN1, IL-18, Neprilysin, Stabilin-2, IL-17 RD, Albumin, Follistatin-like 1, MMP-10, FKBP51, LRRC4, Pref-1, Galectin-1, Troponin C, UNC5H3, FLRT2, CD314, Semaphorin 6B, Netrin-4, CD27 Ligand, IL-20 R beta, Semaphorin 6A, TSK, Cytokeratin-8, CHST3, Mcl-1, DPPIV, SREC-IL, Norrin, JAM-C, Bcl-10, Wnt-4, LSECtin, Kell, TNF RI, PTP1B, htPAPP-A, IDO, PDGF-CC, Galanin, Activin A, TLR2, SCCA2, FABP1, eNOS, SHP-1, ICOS, ClqTNF9, MMP-1, TC-PTP, IL-24, gp130, C-myc, LILRB4, BMP-2, MIA, CD34, CD63, CD9, CD81, IFNab R2, Glypican 2, MSP R, DSCAM, Matriptase, KIR2DL3, CD30, Siglec-10, CLEC-1, TPP1, Ubiquitin+1, ANGPTL4, TWEAK R, Nidogen-1, CD2, Kallikrein 1, TSLP R, LAMP1, TROY, VCAM-1, Siglec-11, S100A1, PAR1, Thyroid Peroxidase, Aminopeptidase P2, IL-1 RI, ADAMS, OSM R beta, Thrombospondin-2, SMPD1, B2M, MFRP, LRP-6, ST3GAL1, NCAM-1 (CD56), Granzyme B, Adiponectin, IL-22BP, TPST2, PD-ECGF, LH, LEDGF, Cyr61, ULBP-3, IFNb, THSD1, FGF-23, LAMA4, Adipsin, AIF, SorCS2, SULT2A1, CD39L2, Insulin R, HIF-1 alpha, OX40 Ligand, Pax3, UCH-L3, cMASP3, Langerin, Desmin, SOX9, ST6GAL1, MEP1B, CD99-L2, Plexin A4, Semaphorin 4D, ROBO2, PDX-1, APRIL, Neurturin, Kremen-2, EMMPRIN, Activin RIB, Neuroligin 2, Epiregulin, CASA, MMP-12, GALNT2, CEACAM-5, VEGF R1, DSPG3, SorCS1, Matrilin-2, sFRP-3, p53, EphB3, NCK1, Semaphorin 7A, NKp80, Prolactin, Cystatin B, Sirtuin 1, FGF-16, FGF R5, NQO-1, Semaphorin 6D, FGF-3, GATA-4, VAP-A, CHST2, Pappalysin-2, Syndecan-3, Jagged 1, AKR1C4, Olfactomedin-2, Osteoadherin, NKp44, Thyroglobulin, IL-21R, Chemerin, EphA1, CD48, MICB, FGF-5, TRANCE, CES2, ULBP-1, Integrin alpha 5, VAMP-2, FLRG, Ret Midkine, CD73, TRACP, proGRP, Granzyme H, PRX2, p2′7, Siglec-6, Dectin-1, CD51, Notch-1, Calreticulin, DR3, DCTN1, CDC25B, Osteoactivin, ACE, CA125, HAO-1, PSMA1, FCRLB, BMP-9, CRIM1, LIF, SPINK1, EphB6, RGM-B, HS3ST1, ROR1, CMG-2, 4-1BB Ligand, LiCAM-2, p63, Cathepsin V, Testican 2, Glypican 5, CD6, Siglec-2, Legumain, PRELP, CES1, TAZ, NSE, TECK, HTRA2, HIF-1 beta, TAFA1, Podocalyxin, RalA, CRELD2, GRAP2, SP-D, BID, GFR alpha-2, Notch-3, VEGF R3, DLL4, TGFb2, LIGHT, XIAP, ST8SIA1, Cathepsin L, 6Ckine, MIS RI, Kallikrein 5, TGM3, FCAR, Contactin-2, CD83, IL-1 R3, SALM4, GBA3, ROBO4, OSCAR, VEGF, IGSF3, Biglycan, Neudesin, ILT4, uPAR, Axl, WIF-1, IL-7 R alpha, GPR56, CEACAM-3, MCEMP1, FABP2, Plexin B3, MEPE, Activin RIIA, ANG-2, Cochlin, Presenilin 1, NPTXR, SLAM, COMT, SPHK1, RBP4, Nectin-1, GUSB, Nidogen-2, IL-17F, SR-AI, TAFA2, N-Cadherin, IL-17B, IL-17 RC, MIP-3b, Cystatin C, Cystatin D, AMSH, FcERI, CLEC10A, HGF R, ANG-1, Prolactin R, FGF-20, CD28, Nogo-A, HSD17B1, IL-19, Enteropeptidase, Cathepsin E, TSLP, TCN2, GDF-15, Epimorphin, GRKS, PD-1, Serpin A4, ADAM23, NOV, Galectin-2, Neurexin 3 beta, TLR3, Sirtuin 2, Numb, IL-28 R alpha, IL-33, Lin28, FCRL1, KLF4, NKp30, Lymphotactin, Cystatin SN, JAM-A, Calreticulin-2, ErbB4, BMP-8, IL-27 Ra, Fas, IL-4 Ra, Kallikrein 14, Matrilin-3, Olig2, Kallikrein 12, CA13, IL-9, Nectin-3, MPIF-1, Cystatin S, ADA, IL-2 Rb, GFR alpha-1, Smad4, ICAM-1, MEF2C, TREM-1, L-Selectin, Hepsin, CD42b, MCSF, RANK, CHST4, CA8, FCRL3, ASAH2, CF XIV, PYY, HGF, I-TAC, Semaphorin 4C, SorCS3, Tie-1, IL-31 RA, Arginase 1, POGLUT1, IL-Ira, Podoplanin, TIM-3, CREG, CD300f, uPA, EphA2, LRRTM4, LIMPII, Tenascin R, CPE, PECAM-1, DNAM-1, DKK-1, OPG, CPB1, TSH, MMP-2, Siglec-9, ICAM-3, Cystatin SA, Galectin-4, Pepsinogen II, Desmoglein-3, Nectin-4, SCF, Serpin A5, PTH, FGF-19, MSP, IL-28A, FGF-12, METAP2, ASAHL, EDIL3, NTAL, EGF R, TAFAS, Galectin-9, vWF-A2, TACE, Activin RIM, Cathepsin S, LDL R, BMPR-IA, OX40, IL-13 R2, B7-H4, MMP-13, ANGPTL7, TRAIL R4, IGSF4B, Sirtuin 5, PEAR1, SH2D1A, Cerberus 1, GDF-11, Nrf2, TROP-2, NUDTS, ROR2, EphB4, Glypican 1, LAP(TGFb1), Gash, Contactin-1, IL-27, UNC5H4, ICAM-2, MBL, HS3ST3B1, RCOR1, IL-10 Rb, XEDAR, IL-22, PILR-alpha, NRG1-131, FABP4, RGM-A, RELT, TrkC, CSa, SREC-I, Nestin, TPO, ErbB3, Kirre13, FLRT1, Galectin-3, CXCL16, JAM-B, DR6, Nogo Receptor, TLR4, VEGF R2, Tie-2, IL-15 R, Caspr2, LTbR, LAMP, ALCAM, GLP-1, NG2, IL-22 R alpha 1, AMIGO2, HCC-1, TFPI-2, ULBP-2, Desmoglein 2, Aggrecan, Syntaxin 4, VAMP-1, Nectin-2, FGF-21, Flt-3, GFAP, TIM-1, Inhibin A, Cadherin-4, PIGF-2, Neurogranin, HE4, IL-23 R, Galectin-7, GALNT3, GITR L, CD14, R-Spondin 2, CK19, Cardiotrophin-1, TREML1, HAPLN1, CD27, ANG-4, Siglec-7, CD155, VEGF-C, TNF RII, PGRP-S, SDF-1a, PDGF-AB, GPVI, CD40, SCF R, Thrombospondin-5, IL-1 RII, Neuropilin-2, Cadherin-13, E-Selectin, GITR, WISP-1, Renin, AgRP, MDL-1, ROBO3, RANTES, Endocan, Granulysin, hCGb, Mesothelin, TLR1, TRAIL, MOG, DDR1, NGF R, TRAIL R3, Trypsin 3, ARSB, LIF R alpha, BAFF R, CD157, Granzyme A, 2B4, ESAM, IL-1 R4, CXCL14, IL-31, SIRP alpha, Uromodulin, CTRC, CEACAM-1, TARC, MIP-3a, SDF-1b, NKp46, MCP-3, IL-32 alpha, TGFb3 FOLR2, CD58, IL-23, CD36, TNFb, Shh-N, Ficolin-1, Reg4, ILT2, Mer, TREM-2, Flt-3L, CDS, IL-6, CD229, Insulin, Syntaxin 6, GRO, Bcl-w, Lipocalin-2, PDGF-AA, IL-2 Ra, Angiogenin, LYVE-1, CD4, RAGE, CDNF, Brevican, NAP-2, PU.1, EDAR, ADAMTS13, Kynureninase, PTH1R, IFN-gamma R1, CrkL, B7-1, PARC, Draxin, VE-Cadherin, Procalcitonin, SOX15, Kallikrein 11, BCMA, Dectin-2, EpCAM, HCC-4, TGFa, IP-10, BLAME, CILP-1, PIGF, LOX-1, MCP-2, Resistin, HVEM, ENPP-7, Syndecan-4, IL-2 Rg, MICA, Dopa Decarboxylase, NPDC-1, MCP-4, EG-VEGF, Glycoprotein V, Semaphorin 4G, IL-12p40, PSA-total, IL-15, MAP1D, C1q, TNF4, Dtk, Endoglin, ENA-78, Reg3A, MIP-1b, FGF-17, IL-6R, IL-8, Galectin-8, CA4, Cystatin E M, FUT8, B7-H3, GCP-2, CD40L, MDC, 4-1BB, HO-1, SOST, S100A13, Kallikrein 7, and IL-13.

Extracellular vesicles (EV) are small membrane bound spheres containing proteins and RNA (of which exosomes are a subset). Exosomes are small (e.g., 20-150 nm) diameter lipid bilayer vesicles secreted by cells to enable paracrine communication. Other EV populations are derived directly from the plasma membrane or are formed during apoptosis (apoptotic bodies). Disclosed herein are compositions comprising a therapeutically effective amount of an MSC secretome (such as, for example, including, but not limited to MSC growth factor, MSC exosome, MSC extracts and/or extracellular vesicle comprising compositions). In some embodiments, the therapeutic composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, or 100 or more of the following nucleic acids, in any combination, or all of the following nucleic acids: hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-1′7-5p, hsa-miR-181a-5p, hsa-miR-191-5p, hsa-miR-193a-5p, hsa-miR-193b-3p, hsa-miR-19′7-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p.

Exemplary microRNA content may include human miRNA sequences hsa-let-7a-5p, hsa-let-7b-5p, hsa-let-7c-5p, hsa-let-7g-5p, hsa-let-7i-5p, hsa-miR-214-3p, and hsa-miR-27a-3p, which all have binding sites in mRNA for TMPRSS2.

In some embodiments, the therapeutic composition comprises extracellular vesicles with a phenotype of CD63⁺ CD9⁻ CD81⁻. In some embodiments, at least 70, 75, 80, 85, 90, 91, 92, 93, 94, or 95% of the extracellular vesicles in the therapeutic composition are CD63⁺ CD9⁻ CD81⁻. In some embodiments, at least 50, 60, 70, 80, 85, 90, 91, 92, 93, 94, or 95% of the extracellular vesicles in the therapeutic composition are CD9⁻. In some embodiments, at least 50, 60, 70, 80, 85, 90, 91, 92, 93, 94, or 95% of the extracellular vesicles in the therapeutic composition are CD81⁻.

In some embodiments, the MSCs cultured to produce the therapeutic composition have the capacity to undergo trilineage differentiation in vitro toward adipocyte, osteoblast, and chondrocyte phenotypes. In some embodiments, the MSCs are positive for CD73, CD105, CD166, and CD90 and are negative for CD14, CD31, CD34, and CD45.

It is understood and herein contemplated that the MSC secretome comprises exosomes and growth factors. The growth factors and exosomes can be allogenic or autogenic. The growth factors and exosomes can be derived from any cell in the human body, such as from ectodermal cells, endodermal cells, or mesodermal cells. For example, the MSC secretomes may comprise mesenchymal stem cell (MSC) derived growth factors, MSC derived exosomes, or both MSC derived growth factors and exosomes. In some embodiments, the method further comprises adding at least one additive with the exosomes and growth factors. Specifically, MSCs under appropriate wound healing conditions may produce suitable therapeutic agents, such as exosomes and growth factors, that can provide therapy for inflammatory lung diseases. In one aspect, disclosed herein are compositions, wherein the MSC secretome composition further comprises prostaglandin E2 (PGE2), transforming growth factor 131 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-a (TGF-a) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF estrogen, and/or thyroid hormones.

Embodiments of a therapeutic composition described herein may comprise proteins and microRNAs, some of which may be embedded in or surrounded by a lipid membrane to create vesicles in the size range of about >20 nm to about 200 nm in size. The number of vesicles within the composition may be between about 1 million to about 100 billion vesicles per mL when suspended or about 10 million to about 1 trillion when formulated as a lyophilized powder.

### A. Pharmaceutical Carriers/Delivery of Pharmaceutical Products

Therapeutic compositions described herein may be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector.

Administration of the compositions by inhalant can be through the nose or mouth via delivery by a spraying or droplet mechanism such as, for example, a metered-dose inhaler, a dry powder inhaler, a nebulizer, a vaporization device, or the like. Delivery can also be directly to any area of the respiratory system (e.g., lungs) via intubation. The exact amount of the compositions required will vary from subject to subject, depending on the species, age, weight and general condition of the subject, the severity of the disorder being treated, mode of administration and the like.

Parenteral administration of the composition, if used, is generally characterized by injection. Injectables can be prepared in conventional forms, either as liquid solutions or suspensions, solid forms suitable for solution of suspension in liquid prior to injection, or as emulsions. Parenteral administration may involve use of a slow release or sustained release system such that a constant dosage is maintained.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution, and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration may include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### B. Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days, and/or one or more dose administrations monthly for two or more months, or once every 1, 2, or 3 or more months. Guidance can be found in the literature for appropriate dosages for given classes of pharmaceutical products.

### C. Mesenchymal Stem Cells and Therapeutic Secretome Compositions

The treatment compositions disclosed herein utilize MSC secretomes and/or growth factors derived from mesenchymal stem cells (MSCs). In one aspect, disclosed herein are MSC secretome compositions (including, but not limited to MSC growth factor, MSC exosome, MSC extracts and/or extracellular vesicle comprising compositions). The treatment compositions may be used in the treatment, inhibition, decrease, reduction, amelioration, and/or prevention of conditions such as, for example, complex regional pain syndrome (CRPS).

MSCs are multipotent cells that have the ability to differentiate into a multitude of cell types including myocytes, chondrocytes, adipocytes, and osteoblasts. Typically, these cells can be found in the placenta, umbilical cord blood, adipose tissue, bone marrow, or amniotic fluid, including perivascular tissue. As used herein, “MSC” refers to non-terminally differentiated cells including but not limited to multipotential stem cell, multipotential stromal cell, stromal vascular cells, pericytes, perivascular cells, stromal cells, pluripotent cells, multipotent cells, adipose-derived fibroblast-like cells, adipose-derived stromal vascular fraction, adipose-derived MSC, bone marrow-derived fibroblast-like cells, bone marrow-derived stromal vascular fraction, bone marrow-derived MSC, tissue-derived fibroblast-like cells, adult stem cells, adult stromal cells, keratinocytes, and/or melanocytes.

MSCs, in addition to their differentiation potential, have immunomodulatory abilities resulting in the expression of many different cytokines and growth factors. As used herein, a “MSC preparation” or “MSC secretome composition” refers to a composition comprising MSC growth factors, MSC exosomes, extracellular vesicles, extracellular vesicle isolate product (EVIP), or acellular extracts of MSCs and/or MSC lysates obtained from human MSCs, fibroblast-like cells, and non-human animal MSCs including, but not limited to MSCs from horses, cows, pigs, sheep, non-human primates, dogs, cats, rabbits, rats, and mice. In embodiments, the MSCs may be derived from the patient to which the composition will be applied (autologous) or derived from another individual (allogeneic). The MSCs may be culture expanded to collect the conditioned media or to increase the quantity of cells for the lysate or used freshly prior to incorporation into the composition of the present disclosure. The MSC secretome compositions (including, but not limited to MSC growth factor, MSC exosome, MSC extracts and/or extracellular vesicle comprising compositions) may comprise about 0.00001 to about 20 wt. %, such as from about 0.01 to about 10 wt. %, of a mesenchymal stem cell (MSC) extract, MSC exosome, or MSC growth factor preparation. The MSC preparation may comprise either MSC conditioned media or MSC lysate from cell culture expanded MSCs. In some embodiments, the composition may further comprise from about 0.01 to about 10 wt. % of a cell-free medium conditioned by growth of MSCs or MSC lineage cells, wherein the cells are cultured under normal hyperoxic culturing conditions or under artificial wound healing conditions.

As disclosed herein the MSCs used to produce the disclosed MSC additives (including growth factor secretome composition either frozen or powdered additives) can be selectively stimulated to produce MSC growth factors, secretomes, cytokines, chemokines, mesenchymal stem cell proteins, peptides, glycosaminoglycans, extracellular matrix (ECM), proteoglycans, secretomes, and exosomes. The growth factors and exosomes may be derived from any cell in the human body, such as from ectodermal cells, endodermal cells, or mesodermal cells. As used herein, MSC growth factors include but are not limited to prostaglandin E2 (PGE2), transforming growth factor 131 (TGF-(31), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-a (TGF-a) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF), as well as hormones including estrogen, and thyroid hormones.

Culturing the MSCs may occur under wound healing and/or hypoxic conditions. Hypoxic conditions may comprise about 1% to about 5% oxygen, reduced or no serum, reduced glucose, or these elements in various combinations. The combined reduced nutrient and metabolite environment may trigger the cultured cells to produce wound healing and anti-inflammatory ECM proteins and growth factors to direct tissue healing. Direct tissue healing likely is in the form of new ECM proteins, such as collagen and glycosaminoglycans (GAGs), as well as growth factors and cytokines. In one aspect, the MSC preparation (such as, for example, a MSC secretome composition) comprises MSC growth factors, MSC exosomes, and/or cellular extracts of MSCs or MSC lysates obtained from MSCs cultured under standard hyperoxic culturing conditions (for example, 21% oxygen) or MSCs cultured under artificial wound healing conditions (such as, for example, 0.1% to about 5% oxygen).

As disclosed herein artificial wound healing conditions simulate growth conditions in real wounds where there is a reduction in nutrient supply and reduction of waste removal that is usually caused by a disruption in local blood circulation. This creates a harsh environment for cells until new blood vessels are created and blood circulation is restored. Accordingly, artificial wound healing conditions used to culture MSCs may include one or more of the following growth conditions reduction in glucose availability, reduction in oxygen tension, reduction in pH, and increased temperature.

In some embodiments, the glucose availability can be reduced relative to normal control (e.g., 4.5 g/L). Modified culture media to reduce glucose, but not damage the cells can be between 0 and 50% reduction in glucose, more preferably between about 5% and 40% reduction in glucose. For example, MSC artificial wound healing culture conditions can comprise glucose reduction of about 5% to about 15%, from about 10% to about 20%, from about 15% to about 25%, from about 20% to about 30%, or from about 25% to about 35%. In some embodiments, glucose is present at a concentration of about 0.1, 0.5, 1.0, 1.5, 2.0, 2.5, 3.0, 3.5, or 4.0 g/L, or a range between any two of these values. In some embodiments, glucose is present at a concentration of less than or no more than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 g/L.

In some embodiments, oxygen tension can be reduced to oxygen levels to hypoxic conditions. Normal atmospheric oxygen is approximately 21% and any reduction is considered hypoxic. Thus, in one aspect, MSCs can be cultured at between 0.0% and 20.9% oxygen, from about 0.1% to about 0.5% oxygen, from about 0.1% to about 2.0%, from about 0.1% to about 5.0% oxygen, from about 0.5% to 5.0%, from about 1.0% to about 10% oxygen, about 5.0% to about 10.0% oxygen; and from about 10.0% to about 15.0%. The hypoxic oxygen conditions may be an aspect of artificial wound healing conditions. Oxygen tension may be between about 0.5% and 20.5% oxygen when culturing MSCs to produce a therapeutic secretome composition comprising extracellular vesicles and/or MSC-secreted growth factors, such as, for example, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, or 20.0% oxygen, or a range between any two of these values.

The pH can also be reduced during MSC culturing. The pH can be from about 6.0 to about 7.4, for example, from 6.0 to about 6.4, from about 6.2 to about 6.4, from about 6.2 to about 6.6, from about 6.4 to about 6.6, from about 6.4 to about 6.8, or from about 6.6 to about 7.0, such as 6.0, 6.1, 6.2, 6.3, 6.4, 6.5, 6.6, 6.7, 6.8, 6.9, 7.0, 7.1, 7.2, 7.3 or 7.4.

The temperature of the culture environment may be raised relative to physiologic homeostasis temperature (e.g., 37° C.). In one aspect, the culture conditions for the MSCs can comprise from about 35° C. to about 39° C., from about 35° C. to about 36° C., from about 36° C. to about 37° C., from about 37° C. to about 38° C., from about 38° C. to about 39° C., from about 39° C. to about 40° C. In one aspect, the temperature of the culture can be 35.0, 35.1, 35.2, 35.3, 36.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, or 40.0° C.

In some embodiments, the culture media is serum free. In some embodiments, the serum free culture media comprises platelet lysate. In some embodiments, the platelet lysate is human platelet lysate (HPL). In some embodiments, the serum free culture media comprises at least 1%, 2%, 3%, 4%, 5%, 6%, 7%, 8%, 9%, 10%, 11%, 12%, 13%, 14%, 15%, 16%, 17%, 18%, 19%, or 20% of HPL by volume, or a range between any two of these values. In some embodiments, the culture media comprises from 8% to 12%, 5% to 15%, or 9% to 11% of HPL by volume.

In one aspect, the MSC secretome compositions (including, but not limited to MSC growth factor, MSC exosome, MSC extracts and/or extracellular vesicle comprising compositions) can further comprise a protective coating (such as, for example, a cryoprotectant oligosaccharide and a protein solution) to reduce degradation of the growth factors. It is understood and herein contemplated that the protective coating can be engineered as a polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc. In one aspect, the gel matrix can comprise copolymers, block copolymers, diblock copolymers, and/or triblock copolymers. In one aspect, the protective coating can comprise a biocompatible polymer. In one aspect, biocompatible polymer can be crosslinked. Such polymers can also serve to slowly release the adipose browning agent and/or fat modulating agent into tissue. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA(m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments the protective coating comprises carbohydrate construction of monosaccharides as well as carbohydrate polymers such as disaccharides or polysaccharides including but not limited to non-reducing poly or disaccharides as well as any combination thereof. Examples of carbohydrates that can be used in the protective coating comprise Glucose, Aldoses (D-Allose, D-Altrose, D-Mannose, etc.), Glucopyranose, Pentahydroxyhexanal, a-D-Glucopyranosyl-D-glucose, a-D-Glucopyranosyl-dihydrate, Polymer of P-D-Glycopyranosyl units, P-D-Fructofuranosyl a-D-glucopyranoside (anhydrous/dihydrate), f3-D-Galactopyranosyl-D-glucose, a-D-Glucopyranosyl-a-D-glucopyranoside (anhydrous/dihydrate), Galactose, Pentoses (Ribose, xylose, lyxose), Dextrose, Dodecacarbon monodecahydrate, Fructose, Sucrose, Lactose, Maltose, Trehalose, Agarose, D-galactosyl-0-(1-4)-anhydro-L-galactosyl, Cellulose, Polymer of P-D-Glycopyranosyl units, and Starch, as well as, Polyhydric alcohols, Polyalcohols, Alditols, Erythritol, Glycitols, Glycerol, Xylitol, and Sorbitol.

In some embodiments the protective coating contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like. Examples of diblock copolymers that can be used in the protective coatings disclosed herein comprise a polymer such as, example, polyethylene glycol (PEG), polyvinyl acetate, polyvinyl alcohol (PVA), polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA).

In one aspect, the protective coating contains (i.e., the encapsulated, the encapsulated compositions can further comprise lecithin or hydrolyzed lecithin as a carrier or as encapsulation material. As used herein, lecithin and/or hydrolyzed lecithin coatings include coatings comprising phosphatidyl choline, phosphatidyl inositol, phosphatidyl ethanolamine, phosphatidylserine, and phosphatidic acid. Sources of the lecithin can be plant or animal sources.

In one aspect, any of the polymers, monosaccharides, disaccharides, or polysaccharides used to form the protective coating formed by placing the MSC additive in an encapsulating solution can be at an appropriate concentration for form the protective coating. For example, polymers, monosaccharides, disaccharides, or polysaccharides can be at any concentration between 0.01 mM and 10.0 M concentration, for example, from about 0.01 M to about 0.1 M, from about 0.1 mM to about 1.0 M, from about 1.0 M to about 10.0 M.

In one aspect, the MSC secretome compositions (including, but not limited to MSC growth factor, MSC exosome, MSC extracts and/or extracellular vesicle comprising compositions) disclosed herein may comprise any known ingredients typically found pharmaceutical fields such as agents for combating free radicals; bactericides; sequestering agents; preservatives; basifying or acidifying agents; fragrances; surfactants; fillers; natural products or extracts of natural product, such as aloe or green tea extract; vitamins; or coloring materials. Other ingredients that may be combined with the powder may include an antioxidant, which can be selected from a variety of antioxidants. Suitable antioxidants include vitamins, such as Vitamin C (L-Ascorbate, Ascorbate-2 Phosphate magnesium salt, Ascorbyl Palmitate, Tetrahexyldecyl Ascorbate), Vitamin E (Tocotrienol), Vitamin A (retinol, retinal, retinoic acid, provitamin A carotenoids, such as beta-carotene), N-acetyl glucosamine, or other derivatives of glucosamine. Other ingredients may include at least one essential fatty acid, such as S2-3, S2-6, and S2-9 polyunsaturated fatty acids, such as linoleic acid (LA), gamma-linoleic acid (GLA), alpha-linoleic acid (ALA), dihomo-y-linolenic acid (DGLA), arachidonic acid (ARA), and others. The fatty acids may be derived from various sources including evening primrose oil, black currant oil, borage oil, or GLA modified safflower seeds. Other ingredients may include a platelet rich fibrin matrix, at least one ingredient to support ECM production and production of hyaluronic acid, such as N-acetyl glucosamine or other derivatives of glucosamine, ultra-low molecular weight (ULMW) hyaluronic acid, chondroitin sulfate, or keratin sulfate.

Producing the MSC secretome compositions can comprise culturing MSCs collected from a donor to create a cultured media under culturing conditions including, in some embodiments, reduced oxygen and nutrition; stimulating the cultured cells to selectively secrete desired anti-inflammatory proteins, peptides, glycosaminoglycans, proteoglycans exosomes, and secretomes by adjusting the cell growth conditions; collecting, combining the conglomerate mixture with an encapsulation solution, and freezing the conglomerate mixture, wherein the conglomerate mixture comprises exosomes, peptides, proteins, cytokines, growth factors, extracellular matrix (ECM), proteoglycans, glycosaminoglycans; and chemokines selected from the group consisting of human MSCs, animal MSCs, multipotential stromal cells, fibroblasts, and fibroblast cells; combining the conglomerate mixture with an encapsulation solution, such as oligosaccharides, like a trehalose solution or protein solution and freezing the mixture; and lyophilizing or freeze-drying the frozen mixture, creating a dry powder. Alternatively, the MSCs may be lysed to collect all of the MSCs from the culture process, creating an extracted lysate; concentrating the extracted lysate and combining the extracted lysate with an encapsulation solution, such as oligosaccharides like a trehalose solution or protein solution and freezing the mixture; and lyophilizing or freeze-drying the frozen mixture, creating a dry powder. The powder contains a highly concentrated collection of analgesic MSC secretomes and exosomes and extracellular matrix components that are specific to anti-inflammation.

The method may also include filter-sterilizing, concentrating, freezing, or freeze drying the MSC conditioned culture medium. Additionally, the MSC culture medium may be combined with a cryoprotectant prior to freezing.

There are various methods for lysing the MSCs. Lysing may be achieved by the addition of a hypotonic solution or repeated freeze-thaw processes to disrupt the cell membranes. Moreover, the cells may be lysed while attached to the culture surface or in suspension. The cells may also be enzymatically released and/or lysed by mechanical homogenization.

Stimulating the MSC to selectively secrete the desired anti-inflammatory proteins, peptides, glycosaminoglycans, proteoglycans, exosomes and secretomes may be achieved by adjusting the cell growth conditions, such as cell confluency, culture media supplements, nutritional supplements, oxygen levels, length of culture in those conditions, cell passage number or combinations of those, and the like.

### III. Methods of Treating Complex Regional Pain Syndrome (CRPS)

In some embodiments, the therapeutic compositions disclosed herein are used in methods of treating complex regional pain syndrome (CRPS) in a subject. Any of the therapeutic compositions described herein may be used in such a method. In some embodiments, subject has chronic intractable pain of one or more upper or lower extremities. In some embodiments, the subject does not experience escalating or changing pain in the 30 days preceding treatment. In some embodiments, the subject experiences in response to treatment one or more of reduced pain, increased energy, reduced fatigue, increased social functioning, increased emotional wellbeing, increased physical functioning, decreased sudomotor changes, decreased edema, decreased allodynia, decreased hypesthesia, improved motion, and/or improved grip strength. In some embodiments, the subject experiences increased range of motion in one or more joints in response to treatment. In some embodiments, the subject experiences decreased pain flare-ups, decreased swelling, and/or decreased burning sensations in response to treatment. In some embodiments, treatment improves hair and/or nail growth in an affected limb of the subject. In some embodiments, the subject experiences a decrease in pain severity and/or pain interference in response to treatment.

In some embodiments, the investigational product of the present disclosure is administered to a CRPS patient in two doses. In some embodiments, 15 mL of investigational product is administered per dose. In some embodiments, the investigational product is administered via intravenous infusion. In some embodiments, the investigational product is administered on separate days. In some embodiments, the investigational product is administered 1, 2, 3, 4, 5, 6, or 7 days apart. In some embodiments, the effects of treatment persist for at least 6 months or at least one year.

Also disclosed herein are methods of treating CRPS in a subject comprising administering to the subject a composition comprising secreted extracellular vesicles that contain a composition that includes any combination of proteins and/or miRNAs, selected from the following: Ferritin, NUP85, LAMP2, GPR115, Serpin F1, OPN, PAI-1, DAPP1, Cathepsin B, Semaphorin 6C, PDGF R alpha, Sortilin, Serpin B6, Dkk-3, Thrombomodulin, PF4, MIF, Periostin, Furin, TIMP-1, Decorin, PCK1, CD99, CD63, CD9, CD81, Transferrin, DcR3, Lumican, TIMP-2, SLITRK5, FAP, Artemin, DPPII, cIAP-1, Pentraxin 3, Visfatin, Neprilysin, Albumin, Galectin-1, UNC5H3, IL-20 R beta, SREC-II, JAM-C, TNF RI, htPAPP-A, eNOS, MSP R, TPP1, LAMP1, B2M, NCAM-1, HIF-1 alpha, ST6GAL1, CD99-L2, Plexin A4, EMMPRIN, p53, Semaphorin 7A, NKp80, Cystatin B, Osteoadherin, Midkine, Calreticulin, Osteoactivin, Legumain, TAZ, Cathepsin L, RBP4, Serpin A4, JAM-A, MCSF, LIMPII, OPG, IL-22, Galectin-3, MOG, Trypsin 3, SIRP alpha, and Syndecan-4, and at least one protein selected from the group consisting of: Ferritin, IGFBP-4 IL-1 R6 GSTM1, NUP85, LAMP2, MeprinA, IL-1 F10, bIG-H3, GPR115, TGFb1, Ephrin-A4, CD109, Serpin F1, IGFBP-6, HS3ST4, Aminopeptidase LRAP, OPN, PAI-1, DAPP1, GDF-9, Cathepsin B, IGFBP-2, Semaphorin 6C, IGF-2, PDGF R alpha, Sortilin, Serpin B6, Dkk-3, CNTF, TSP-1, GM-CSF Ra, Thrombomodulin, Endoglycan, IGFBP-3, RGM-C, PF4, MIF, TGM4, Periostin, Furin, TIMP-1, PAPP-A, Decorin, PCK1, Arylsulfatase A, CD99, CA2, PRDX4, Transferrin, DcR3, GP73, LAIR2, ULBP-4, Lumican, TIMP-2, TFPI, SOX2, SLITRK5, FAP, Spinesin, ENPP-2, CD97,

CTACK, Integrin alpha 1, EXTL3, IL-18 BPa, PD-L2, PSMA, IL-20 Ra, Glyoxalase II, Trypsin 1, IGF-2R, ADAMTSL-1, Erythropoietin, Plexin D1, DNMT3A, BCL-2, CL-P1, Ephrin-B3, FABP6, CHI3L1, FCRLS, TFF3, Artemin, DPPII, cIAP-1, PDGF Rb, Pentraxin 3, Angiotensinogen, Follistatin, CF VII, Persephin, TRAIL R1, THAP11, CD200, CLEC-2, AMIGO, IGFBP-5, PON1, SOX7, GALNT10, Visfatin, Progranulin, PCSK2, GKN1, IL-18, Neprilysin, Stabilin-2, IL-17 RD, Albumin, Follistatin-like 1, MMP-10, FKBP51, LRRC4, Pref-1, Galectin-1, Troponin C, UNC5H3, FLRT2, CD314, Semaphorin 6B, Netrin-4, CD27 Ligand, IL-20 R beta, Semaphorin 6A, TSK, Cytokeratin-8, CHST3, Mcl-1, DPPIV, SREC-II, Norrin, JAM-C, Bcl-10, Wnt-4, LSECtin, Kell, TNF RI, PTP1B, htPAPP-A, IDO, PDGF-CC, Galanin, Activin A, TLR2, SCCA2, FABP1, eNOS, SHP-1, ICOS, ClqTNF9, MMP-1, TC-PTP, IL-24, gp130, C-myc, LILRB4, BMP-2, MIA, CD34, CD63, CD9, CD81, IFNab R2, Glypican 2, MSP R, DSCAM, Matriptase, KIR2DL3, CD30, Siglec-10, CLEC-1, TPP1, Ubiquitin+1, ANGPTL4, TWEAK R, Nidogen-1, CD2, Kallikrein 1, TSLP R, LAMP1, TROY, VCAM-1, Siglec-11, S100A1, PAR1, Thyroid Peroxidase, Aminopeptidase P2, IL-1 RI, ADAMS, OSM R beta, Thrombospondin-2, SMPD1, B2M, MFRP, LRP-6, ST3GAL1, NCAM-1 (CD56), Granzyme B, Adiponectin, IL-22BP, TPST2, PD-ECGF, LH, LEDGF, Cyr61, ULBP-3, IFNb, THSD1, FGF-23, LAMA4, Adipsin, AIF, SorCS2, SULT2A1, CD39L2, Insulin R, HIF-1 alpha, OX40 Ligand, Pax3, UCH-L3, cMASP3, Langerin, Desmin, SOX9, ST6GAL1, MEP1B, CD99-L2, Plexin A4, Semaphorin 4D, ROBO2, PDX-1, APRIL, Neurturin, Kremen-2, EMMPRIN, Activin RIB, Neuroligin 2, Epiregulin, CASA, MMP-12, GALNT2, CEACAM-5, VEGF R1, DSPG3, SorCS1, Matrilin-2, sFRP-3, p53, EphB3, NCK1, Semaphorin 7A, NKp80, Prolactin, Cystatin B, Sirtuin 1, FGF-16, FGF R5, NQO-1, Semaphorin 6D, FGF-3, GATA-4, VAP-A, CHST2, Pappalysin-2, Syndecan-3, Jagged 1, AKR1C4, Olfactomedin-2, Osteoadherin, NKp44, Thyroglobulin, IL-21R, Chemerin, EphA1, CD48, MICB, FGF-5, TRANCE, CES2, ULBP-1, Integrin alpha 5, VAMP-2, FLRG, Ret Midkine, CD73, TRACP, proGRP, Granzyme H, PRX2, p2′7, Siglec-6, Dectin-1, CD51, Notch-1, Calreticulin, DR3, DCTN1, CDC25B, Osteoactivin, ACE, CA125, HAO-1, PSMA1, FCRLB, BMP-9, CRIM1, LIF, SPINK1, EphB6, RGM-B, HS3ST1, ROR1, CMG-2, 4-1BB Ligand, LiCAM-2, p63, Cathepsin V, Testican 2, Glypican 5, CD6, Siglec-2, Legumain, PRELP, CES1, TAZ, NSE, TECK, HTRA2, HIF-1 beta, TAFA1, Podocalyxin, RalA, CRELD2, GRAP2, SP-D, BID, GFR alpha-2, Notch-3, VEGF R3, DLL4, TGFb2, LIGHT, XIAP, ST8SIA1, Cathepsin L, 6Ckine, MIS RII, Kallikrein 5, TGM3, FCAR, Contactin-2, CD83, IL-1 R3, SALM4, GBA3, ROBO4, OSCAR, VEGF, IGSF3, Biglycan, Neudesin, ILT4, uPAR, Axl, WIF-1, IL-7 R alpha, GPR56, CEACAM-3, MCEMP1, FABP2, Plexin B3, MEPE, Activin RIIA, ANG-2, Cochlin, Presenilin 1, NPTXR, SLAM, COMT, SPHK1, RBP4, Nectin-1, GUSB, Nidogen-2, IL-17F, SR-AI, TAFA2, N-Cadherin, IL-17B, IL-17 RC, MIP-3b, Cystatin C, Cystatin D, AMSH, FcERI, CLEC10A, HGF R, ANG-1, Prolactin R, FGF-20, CD28, Nogo-A, HSD17B1, IL-19, Enteropeptidase, Cathepsin E, TSLP, TCN2, GDF-15, Epimorphin, GRKS, PD-1, Serpin A4, ADAM23, NOV, Galectin-2, Neurexin 3 beta, TLR3, Sirtuin 2, Numb, IL-28 R alpha, IL-33, Lin28, FCRL1, KLF4, NKp30, Lymphotactin, Cystatin SN, JAM-A, Calreticulin-2, ErbB4, BMP-8, IL-27 Ra, Fas, IL-4 Ra, Kallikrein 14, Matrilin-3, Olig2, Kallikrein 12, CA13, IL-9, Nectin-3, MPIF-1, Cystatin S, ADA, IL-2 Rb, GFR alpha-1, Smad4, ICAM-1, MEF2C, TREM-1, L-Selectin, Hepsin, CD42b, MCSF, RANK, CHST4, CA8, FCRL3, ASAH2, CF XIV, PYY, HGF, I-TAC, Semaphorin 4C, SorCS3, Tie-1, IL-31 RA, Arginase 1, POGLUT1, IL-Ira, Podoplanin, TIM-3, CREG, CD300f, uPA, EphA2, LRRTM4, LIMPII, Tenascin R, CPE, PECAM-1, DNAM-1, DKK-1, OPG, CPB1, TSH, MMP-2, Siglec-9, ICAM-3, Cystatin SA, Galectin-4, Pepsinogen II, Desmoglein-3, Nectin-4, SCF, Serpin A5, PTH, FGF-19, MSP, IL-28A, FGF-12, METAP2, ASAHL, EDIL3, NTAL, EGF R, TAFA5, Galectin-9, vWF-A2, TACE, Activin RUB, Cathepsin S, LDL R, BMPR-IA, OX40, IL-3 R2, B7-H4, MMP-13, ANGPTL7, TRAIL R4, IGSF4B, Sirtuin 5, PEAR1, SH2D1A, Cerberus 1, GDF-11, Nrf2, TROP-2, NUDT5, ROR2, EphB4, Glypican 1, LAP(TGFb1), Gash, Contactin-1, IL-27, UNC5H4, ICAM-2, MBL, HS3ST3B1, RCOR1, IL-10 Rb, XEDAR, IL-22, PILR-alpha, NRG1-b1, FABP4, RGM-A, RELT, TrkC, C5a, SREC-I, Nestin, TPO, ErbB3, Kirre13, FLRT1, Galectin-3, CXCL16, JAM-B, DR6, Nogo Receptor, TLR4, VEGF R2, Tie-2, IL-15 R, Caspr2, LTbR, LAMP, ALCAM, GLP-1, NG2, IL-22 R alpha 1, AMIGO2, HCC-1, TFPI-2, ULBP-2, Desmoglein 2, Aggrecan, Syntaxin 4, VAMP-1, Nectin-2, FGF-21, Flt-3, GFAP, TIM-1, Inhibin A, Cadherin-4, PIGF-2, Neurogranin, HE4, IL-23 R, Galectin-7, GALNT3, GITR L, CD14, R-Spondin 2, CK19, Cardiotrophin-1, TREML1, HAPLN1, CD27, ANG-4, Siglec-7, CD155, VEGF-C, TNF RH, PGRP-S, SDF-1a, PDGF-AB, GPVI, CD40, SCF R, Thrombospondin-5, IL-1 MI, Neuropilin-2, Cadherin-13, E-Selectin, GITR, WISP-1, Renin, AgRP, MDL-1, ROBO3, RANTES, Endocan, Granulysin, hCGb, Mesothelin, TLR1, TRAIL, MOG, DDR1, NGF R, TRAIL R3, Trypsin 3, ARSB, LIF R alpha, BAFF R, CD157, Granzyme A, 2B4, ESAM, IL-1 R4, CXCL14, IL-31, SIRP alpha, Uromodulin, CTRC, CEACAM-1, TARC, MIP-3a, SDF-1b, NKp46, MCP-3, IL-32 alpha, TGFb3 FOLR2, CD58, IL-23, CD36, TNFb, Shh-N, Ficolin-1, Reg4, ILT2, Mer, TREM-2, Flt-3L, CDS, IL-6, CD229, Insulin, Syntaxin 6, GRO, Bcl-w, Lipocalin-2, PDGF-AA, IL-2 Ra, Angiogenin, LYVE-1, CD4, RAGE, CDNF, Brevican, NAP-2, PU.1, EDAR, ADAMTS13, Kynureninase, PTH1R, IFN-gamma R1, CrkL, B7-1, PARC, Draxin, VE-Cadherin, Procalcitonin, SOX15, Kallikrein 11, BCMA, Dectin-2, EpCAM, HCC-4, TGFa, IP-10, BLAME, CILP-1, PIGF, LOX-1, MCP-2, Resistin, HVEM, ENPP-7, Syndecan-4, IL-2 Rg, MICA, Dopa Decarboxylase, NPDC-1, MCP-4, EG-VEGF, Glycoprotein V, Semaphorin 4G, IL-12p40, PSA-total, IL-15, MAP1D, C1q, TNF4, Dtk, Endoglin, ENA-78, Reg3A, MIP-1b, FGF-17, IL-6R, IL-8, Galectin-8, CA4, Cystatin E M, FUT8, B7-H3, GCP-2, CD40L, MDC, 4-1BB, HO-1, SOST, S100A13, Kallikrein 7, IL-13, hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-1′7-5p, hsa-miR-181a-5p, hsa-miR-191-5p, hsa-miR-193a-5p, hsa-miR-193b-3p, hsa-miR-19′7-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p.

In some embodiments, the therapeutic product is administered at a cell-equivalent dose range of 0.7 to 7 million cells/kg. In some embodiments, the therapeutic product is administered at a cell-equivalent dose of at least about, at most about, or about 0.2, 0.5, 0.7, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, or 12.0 million cells/kg, or a range between any two of these values. In some embodiments, the product is administered at a dose that provides 9×10¹¹ to 1.2×10¹² extracellular vesicles or 5×10¹¹ to 1.5×10¹², 6×10¹¹ to 1.4×10¹², 7×10¹¹ to 1.3×10¹², 8×10¹¹ to 1.2×10¹², or 8×10¹¹ to 1.3×10¹² extracellular vesicles. In some embodiments, the product is administered at a dose that provides at least or at most 5×10¹¹, 6×10¹¹, 7×10¹¹, 8×10¹¹, 9×10¹¹, 1×10¹², 1.1×10¹², 1.2×10¹², 1.3×10¹², 1.4×10¹², or 1.5×10¹² extracellular vesicles. In some embodiments, the therapeutic product comprises 6×10¹⁰ to 8×10¹⁰, 5×10¹⁰ to 9×10¹⁰, 4×10¹⁰ to 10×10¹⁰, 5.5×10¹⁰ to 8.5×10¹⁰, or 6×10¹⁰ to 8.5×10¹⁰ cells/ml. In some embodiments, the therapeutic product comprises 6×10¹⁰ to 8×10¹⁰ extracellular vesicles or cells per ml and is administered at a dose of 10 to 20 ml. In some embodiments, the therapeutic product comprises 6×10¹⁰ to 8×10¹⁰ extracellular vesicles or cells per ml and is administered at a dose of 10, 11, 12, 13, 14, 15, 16, 17, 18, 19, or 20 ml, or a range between any two of these values.

In some embodiments, the subject has a decrease of at least 50% in a Visual Analog Scale of Pain (VAS) score after administration compared to before administration. For example, the subject can have a decrease of at least 50%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% in a VAS score after administration compared to before administration. In some embodiments, the subject has a minimum VAS score of greater than 6 in an affected extremity prior to administration.

In some embodiments, the subject has a decrease in a Brief Pain Inventory (BPI) score after administration compared to before administration. In some embodiments, the subject has a decrease in a BPI score of at least 10%, at least 20%, at least 30%, or greater than 30% after administration compared to before administration. For example, the subject can have a decrease in a BPI score of at least 10%, 11%, 12%, 13%, 14%, 15%, 16%, 17%, 18%, 19%, 20%, 21%, 22%, 23%, 24%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% after administration compared to before administration. In some embodiments, the subject has a decrease in a BPI pain interference score of at least 10%, at least 20%, at least 30%, or greater than 30% after administration compared to before administration. For example, the subject can have a decrease in a BPI pain interference score of at least 10%, 11%, 12%, 13%, 14%, 15%, 16%, 17%, 18%, 19%, 20%, 21%, 22%, 23%, 24%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% after administration compared to before administration.

In some embodiments, the subject has an increase in a range of motion assessment score of at least 50%, at least 60%, at least 70%, at least 75%, at least 80%, at least 85%, at least 95%, or at least 100% after administration compared to before administration. For example, the subject can have an increase in a range of motion assessment score of at least 50%, 50%, 51%, 52%, 53%, 54%, 55%, 56%, 57%, 58%, 59, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or at least 100% after administration compared to before administration.

In some embodiments, the subject has an increase in a Jamar dynamometer test assessment score after administration as compared to before administration. In some embodiments, the increase in the Jamar dynamometer test assessment score is in an upper extremity. In some embodiments, the subject has an equalization of grip strength after administration. In some embodiments, the subject has an increase in grip strength of at least 5 kg, at least 10 kg, at least 15 kg, at or least 20 kg after administration compared to before administration. In some embodiments, the subject has an increase in grip strength of at least 50%, at least 75%, at least 100%, at least 125%, at least 150%, at least 175%, at least 200%, or at least 210% after administration compared to before administration. For example, the subject can have an increase in grip strength of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 210%, 220%, 230%, 240%, 250%, 260%, 270%, 280%, 290%, or at least 300%, after administration compared to before administration. In some embodiments, the subject has an increase in grip strength in an affected limb. In some embodiments, the subject has an increase in grip strength in a control limb. In some embodiments, the subject has an improvement after administration compared to before administration in one or more of: limb range of motion, joint range of motion, sudomotor changes, edema, allodynia, and hypesthesia.

In some embodiments, the subject has an improvement in an Short Form 36 Health Survey Questionnaire (SF-36) Questionnaire score after administration compared to before administration. In some embodiments, the improvement in the SF-36 Questionnaire score comprises one or more of: an improvement in pain, an improvement in energy levels, an improvement in social functioning, an improvement in physical functioning, a positive trend in emotional wellbeing, and an improvement in general health. In some embodiments, the subject has chronic intractable pain of one or more upper or lower extremities. In some embodiments, the subject does not experience escalating or changing pain in the 30 days preceding administration. In some embodiments, the subject experiences decreased pain flare-ups, decreased swelling, and/or decreased burning sensations in an affected limb after administration compared to before administration. In some embodiments, the administration improves hair and/or nail growth in an affected limb of the subject.

In some embodiments, a subject that can be treated with the therapeutic product and methods described herein can comprise any subject suffering from or diagnosed with CRPS. In some embodiments, a subject can comprise a mammal. In some embodiments, a subject can comprise a human. In some embodiments, a subject can comprise a non-human mammal. Non-limiting examples of a non-human mammal can include a non-human primate such as chimpanzee, and other apes and monkey species; farm animals such as cattle, horses, sheep, goats, swine; domestic animals such as rabbits, dogs, and cats; laboratory animals including rodents, such as rats, mice and guinea pigs, and the like.

### IV. Examples

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. These examples are provided for illustrative purposes only and not to limit the scope of the claims provided herein.

### A. Example 1—Production of Therapeutic Composition

An MSC secretome therapeutic composition was made by the following method: human bone marrow-derived MSCs were cultured in culture vessels with growth media to expand the MSC population. Growth media was then removed, and the cells were washed with PBS. The MSCs were then cultured in reduced glucose media with a pH below 7.0 under hypoxic conditions. The conditioned media was then collected and subjected to diafiltration followed by filter sterilization. The production process for the therapeutic product was done under current Good Manufacturing Practices and Current Good Tissue Practices.

The tetraspanin profile of extracellular vesicles present in the therapeutic composition was determined, and it was found that greater than 95% of the extracellular vesicles present in the therapeutic composition were CD63⁺ CD9⁻ CD81⁻.

Protein content of the therapeutic product was determined, and the following proteins were found to be present: Ferritin, NUP85, LAMP2, GPR115, Serpin F1, OPN, PAI-1, DAPP1, Cathepsin B, Semaphorin 6C, PDGF R alpha, Sortilin, Serpin B6, Dkk-3, Thrombomodulin, PF4, MIF, Periostin, Furin, TIMP-1, Decorin, PCK1, CD99, CD63, CD9, CD81, Transferrin, DcR3, Lumican, TIMP-2, SLITRK5, FAP, Artemin, DPPII, cIAP-1, Pentraxin 3, Visfatin, Neprilysin, Albumin, Galectin-1, UNC5H3, IL-20 R beta, SREC-II, JAM-C, TNF RI, htPAPP-A, eNOS, MSP R, TPP1, LAMP1, B2M, NCAM-1, HIF-1 alpha, ST6GAL1, CD99-L2, Plexin A4, EMMPRIN, p53, Semaphorin 7A, NKp80, Cystatin B, Osteoadherin, Midkine, Calreticulin, Osteoactivin, Legumain, TAZ, Cathepsin L, RBP4, Serpin A4, JAM-A, MCSF, LIMPII, OPG, IL-22, Galectin-3, MOG, Trypsin 3, SIRP alpha, and Syndecan-4, and at least one protein selected from the following: Ferritin, IGFBP-4 IL-1 R6 GSTM1, NUP85, LAMP2, MeprinA, IL-1 F10, bIG-H3, GPR115, TGFb1, Ephrin-A4, CD109, Serpin F1, IGFBP-6, HS3ST4, Aminopeptidase LRAP, OPN, PAI-1, DAPP1, GDF-9, Cathepsin B, IGFBP-2, Semaphorin 6C, IGF-2, PDGF R alpha, Sortilin, Serpin B6, Dkk-3, CNTF, TSP-1, GM-CSF Ra, Thrombomodulin, Endoglycan, IGFBP-3, RGM-C, PF4, MIF, TGM4, Periostin, Furin, TIMP-1, PAPP-A, Decorin, PCK1, Arylsulfatase A, CD99, CA2, PRDX4, Transferrin, DcR3, GP73, LAIR2, ULBP-4, Lumican, TIMP-2, TFPI, SOX2, SLITRK5, FAP, Spinesin, ENPP-2, CD97, CTACK, Integrin alpha 1, EXTL3, IL-18 BPa, PD-L2, PSMA, IL-20 Ra, Glyoxalase II, Trypsin 1, IGF-2R, ADAMTSL-1, Erythropoietin, Plexin D1, DNMT3A, BCL-2, CL-P1, Ephrin-B3, FABP6, CHI3L1, FCRLS, TFF3, Artemin, DPPII, cIAP-1, PDGF Rb, Pentraxin 3, Angiotensinogen, Follistatin, CF VII, Persephin, TRAIL R1, THAP11, CD200, CLEC-2, AMIGO, IGFBP-5, PON1, SOX7, GALNT10, Visfatin, Progranulin, PCSK2, GKN1, IL-18, Neprilysin, Stabilin-2, IL-17 RD, Albumin, Follistatin-like 1, MMP-10, FKBP51, LRRC4, Pref-1, Galectin-1, Troponin C, UNC5H3, FLRT2, CD314, Semaphorin 6B, Netrin-4, CD27 Ligand, IL-20 R beta, Semaphorin 6A, TSK, Cytokeratin-8, CHST3, Mcl-1, DPPIV, SREC-II, Norrin, JAM-C, Bcl-10, Wnt-4, LSECtin, Kell, TNF RI, PTP1B, htPAPP-A, IDO, PDGF-CC, Galanin, Activin A, TLR2, SCCA2, FABP1, eNOS, SHP-1, ICOS, ClqTNF9, MMP-1, TC-PTP, IL-24, gp130, C-myc, LILRB4, BMP-2, MIA, CD34, CD63, CD9, CD81, IFNab R2, Glypican 2, MSP R, DSCAM, Matriptase, KIR2DL3, CD30, Siglec-10, CLEC-1, TPP1, Ubiquitin+1, ANGPTL4, TWEAK R, Nidogen-1, CD2, Kallikrein 1, TSLP R, LAMP1, TROY, VCAM-1, Siglec-11, S100A1, PAR1, Thyroid Peroxidase, Aminopeptidase P2, IL-1 RI, ADAMS, OSM R beta, Thrombospondin-2, SMPD1, B2M, MFRP, LRP-6, ST3GAL1, NCAM-1 (CD56), Granzyme B, Adiponectin, IL-22BP, TPST2, PD-ECGF, LH, LEDGF, Cyr61, ULBP-3, IFNb, THSD1, FGF-23, LAMA4, Adipsin, AIF, SorCS2, SULT2A1, CD39L2, Insulin R, HIF-1 alpha, OX40 Ligand, Pax3, UCH-L3, cMASP3, Langerin, Desmin, SOX9, ST6GAL1, MEP1B, CD99-L2, Plexin A4, Semaphorin 4D, ROBO2, PDX-1, APRIL, Neurturin, Kremen-2, EMMPRIN, Activin RIB, Neuroligin 2, Epiregulin, CASA, MMP-12, GALNT2, CEACAM-5, VEGF R1, DSPG3, SorCS1, Matrilin-2, sFRP-3, p53, EphB3, NCK1, Semaphorin 7A, NKp80, Prolactin, Cystatin B, Sirtuin 1, FGF-16, FGF R5, NQO-1, Semaphorin 6D, FGF-3, GATA-4, VAP-A, CHST2, Pappalysin-2, Syndecan-3, Jagged 1, AKR1C4, Olfactomedin-2, Osteoadherin, NKp44, Thyroglobulin, IL-21R, Chemerin, EphA1, CD48, MICB, FGF-5, TRANCE, CES2, ULBP-1, Integrin alpha 5, VAMP-2, FLRG, Ret Midkine, CD73, TRACP, proGRP, Granzyme H, PRX2, p2′7, Siglec-6, Dectin-1, CD51, Notch-1, Calreticulin, DR3, DCTN1, CDC25B, Osteoactivin, ACE, CA125, HAO-1, PSMA1, FCRLB, BMP-9, CRIM1, LIF, SPINK1, EphB6, RGM-B, HS3ST1, ROR1, CMG-2, 4-1BB Ligand, LiCAM-2, p63, Cathepsin V, Testican 2, Glypican 5, CD6, Siglec-2, Legumain, PRELP, CES1, TAZ, NSE, TECK, HTRA2, HIF-1 beta, TAFA1, Podocalyxin, RalA, CRELD2, GRAP2, SP-D, BID, GFR alpha-2, Notch-3, VEGF R3, DLL4, TGFb2, LIGHT, XIAP, ST8SIA1, Cathepsin L, 6Ckine, MIS RI, Kallikrein 5, TGM3, FCAR, Contactin-2, CD83, IL-1 R3, SALM4, GBA3, ROBO4, OSCAR, VEGF, IGSF3, Biglycan, Neudesin, ILT4, uPAR, Axl, WIF-1, IL-7 R alpha, GPR56, CEACAM-3, MCEMP1, FABP2, Plexin B3, MEPE, Activin RIIA, ANG-2, Cochlin, Presenilin 1, NPTXR, SLAM, COMT, SPHK1, RBP4, Nectin-1, GUSB, Nidogen-2, IL-17F, SR-AI, TAFA2, N-Cadherin, IL-17B, IL-17 RC, MIP-3b, Cystatin C, Cystatin D, AMSH, FcERI, CLEC10A, HGF R, ANG-1, Prolactin R, FGF-20, CD28, Nogo-A, HSD17B1, IL-19, Enteropeptidase, Cathepsin E, TSLP, TCN2, GDF-15, Epimorphin, GRKS, PD-1, Serpin A4, ADAM23, NOV, Galectin-2, Neurexin 3 beta, TLR3, Sirtuin 2, Numb, IL-28 R alpha, IL-33, Lin28, FCRL1, KLF4, NKp30, Lymphotactin, Cystatin SN, JAM-A, Calreticulin-2, ErbB4, BMP-8, IL-27 Ra, Fas, IL-4 Ra, Kallikrein 14, Matrilin-3, Olig2, Kallikrein 12, CA13, IL-9, Nectin-3, MPIF-1, Cystatin S, ADA, IL-2 Rb, GFR alpha-1, Smad4, ICAM-1, MEF2C, TREM-1, L-Selectin, Hepsin, CD42b, MCSF, RANK, CHST4, CA8, FCRL3, ASAH2, CF XIV, PYY, HGF, I-TAC, Semaphorin 4C, SorCS3, Tie-1, IL-31 RA, Arginase 1, POGLUT1, IL-Ira, Podoplanin, TIM-3, CREG, CD300f, uPA, EphA2, LRRTM4, LIMPII, Tenascin R, CPE, PECAM-1, DNAM-1, DKK-1, OPG, CPB1, TSH, MMP-2, Siglec-9, ICAM-3, Cystatin SA, Galectin-4, Pepsinogen II, Desmoglein-3, Nectin-4, SCF, Serpin A5, PTH, FGF-19, MSP, IL-28A, FGF-12, METAP2, ASAHL, EDIL3, NTAL, EGF R, TAFAS, Galectin-9, vWF-A2, TACE, Activin RIM, Cathepsin S, LDL R, BMPR-IA, OX40, IL-13 R2, B7-H4, MMP-13, ANGPTL7, TRAIL R4, IGSF4B, Sirtuin 5, PEAR1, SH2D1A, Cerberus 1, GDF-11, Nrf2, TROP-2, NUDTS, ROR2, EphB4, Glypican 1, LAP(TGFb1), Gash, Contactin-1, IL-27, UNC5H4, ICAM-2, MBL, HS3ST3B1, RCOR1, IL-10 Rb, XEDAR, IL-22, PILR-alpha, NRG1-131, FABP4, RGM-A, RELT, TrkC, CSa, SREC-I, Nestin, TPO, ErbB3, Kirre13, FLRT1, Galectin-3, CXCL16, JAM-B, DR6, Nogo Receptor, TLR4, VEGF R2, Tie-2, IL-15 R, Caspr2, LTbR, LAMP, ALCAM, GLP-1, NG2, IL-22 R alpha 1, AMIGO2, HCC-1, TFPI-2, ULBP-2, Desmoglein 2, Aggrecan, Syntaxin 4, VAMP-1, Nectin-2, FGF-21, Flt-3, GFAP, TIM-1, Inhibin A, Cadherin-4, PIGF-2, Neurogranin, HE4, IL-23 R, Galectin-7, GALNT3, GITR L, CD14, R-Spondin 2, CK19, Cardiotrophin-1, TREML1, HAPLN1, CD27, ANG-4, Siglec-7, CD155, VEGF-C, TNF RI, PGRP-S, SDF-1a, PDGF-AB, GPVI, CD40, SCF R, Thrombospondin-5, IL-1 RI, Neuropilin-2, Cadherin-13, E-Selectin, GITR, WISP-1, Renin, AgRP, MDL-1, ROBO3, RANTES, Endocan, Granulysin, hCGb, Mesothelin, TLR1, TRAIL, MOG, DDR1, NGF R, TRAIL R3, Trypsin 3, ARSB, LIF R alpha, BAFF R, CD157, Granzyme A, 2B4, ESAM, IL-1 R4, CXCL14, IL-31, SIRP alpha, Uromodulin, CTRC, CEACAM-1, TARC, MIP-3a, SDF-1b, NKp46, MCP-3, IL-32 alpha, TGFb3 FOLR2, CD58, IL-23, CD36, TNFb, Shh-N, Ficolin-1, Reg4, ILT2, Mer, TREM-2, Flt-3L, CDS, IL-6, CD229, Insulin, Syntaxin 6, GRO, Bcl-w, Lipocalin-2, PDGF-AA, IL-2 Ra, Angiogenin, LYVE-1, CD4, RAGE, CDNF, Brevican, NAP-2, PU.1, EDAR, ADAMTS13, Kynureninase, PTH1R, IFN-gamma R1, CrkL, B7-1, PARC, Draxin, VE-Cadherin, Procalcitonin, SOX15, Kallikrein 11, BCMA, Dectin-2, EpCAM, HCC-4, TGFa, IP-10, BLAME, CILP-1, PIGF, LOX-1, MCP-2, Resistin, HVEM, ENPP-7, Syndecan-4, IL-2 Rg, MICA, Dopa Decarboxylase, NPDC-1, MCP-4, EG-VEGF, Glycoprotein V, Semaphorin 4G, IL-12p40, PSA-total, IL-15, MAP1D, C1q, TNF4, Dtk, Endoglin, ENA-78, Reg3A, MIP-1b, FGF-17, IL-6R, IL-8, Galectin-8, CA4, Cystatin E M, FUT8, B7-H3, GCP-2, CD40L, MDC, 4-1BB, HO-1, SOST, S100A13, Kallikrein 7, and IL-13.

The nucleic acid content of the therapeutic product was determined, and the following nucleic acids were found to be present: hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-1′7-5p, hsa-miR-181a-5p, hsa-miR-191-5p, hsa-miR-193a-5p, hsa-miR-193b-3p, hsa-miR-19′7-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p.

### B. Example 2—Safety Study for Treatment of Complex Regional Pain Syndrome (CRPS) Using Human Bone Marrow Stem Cell Derived Extracellular Vesicle Investigational Product

In this Example, the inventors report that a human bone marrow stem cell derived extracellular vesicle (hBM-MSC EV) investigational product (IP) is safe and exhibits efficacy in complex regional pain syndrome patients. Ten subjects received two intravenous infusions, each containing 15 mL of investigational product, on Day 1 and Day 4. Assessments (Visual Analog Scale of Pain, Brief Pain Inventory, SF-36 Questionnaire, Range of Motion Analysis, Jamar dynamometer) were performed at baseline (Day 0, prior to infusion), Week 1, and months 1, 3, and 6 after the second infusion. No serious adverse events related to treatment with the investigational product occurred. Statistically significant improvements in pain and motion assessments occurred across the subject pool. It was found that the investigational product is safe in patients with CRPS. The investigational product exhibited efficacy in addressing CRPS symptoms. The investigational product presents a safe and effective potential treatment for CRPS.

ExoFlo™, the investigational product (IP) used herein, is a human BM-MSC derived EV (hBM-MSC EV) preparation. The IP is a consistent EV product with extensive characterization including advanced particle analysis, proteomic evaluation and USP<71> sterility assurance. Additionally, through manufacturing in a cGMP environment, the IP is a quality bio-pharmacological product that is consistent regarding dose and biological activity. Two peer reviewed studies have demonstrated the safety of administering IV infusions of the IP to severe COVID-19 patients. The IP has also demonstrated efficacy in a subset of these severely ill patients.

### Methods

10 subjects with CRPS who met all inclusion and exclusion criteria received 15 mL of BM-MSC investigational product via intravenous route (IV) over 30 minutes on Day 1 and Day 4 (two doses). 15 mL of the investigational product was mixed with 85 mL of normal saline and the total 100 mL mixture was administered via IV. This dosing protocol was selected as it was previously shown to be safe. Measurements were taken at baseline prior to any dose, one week after the second dose, one month, three months, and six months after the second dose. During the trial, one subject was lost to follow up and their information was excluded from the study results and analyses.

Measurements and Statistical Analyses Assessments performed included the Visual Analog Scale (VAS) of Pain, the Brief Pain Inventory, the SF-36 Questionnaire, Range of Motion (ROM) Analysis and the Jamar dynamometer test. All results were subjected to one-way ANOVA, using the Geisser-Greenhouse correction, followed by Dunnett multiple comparison test using GraphPad Prism 9.5.1.

Study Design. As CRPS affects more women than men (ratio range 2:3-4:1), females were well represented as shown in Table 1. Diversity was not addressed in this pilot study.

TABLE 1
Gender and age demographics of subjects

| Patient Identifier | Age | Gender |
|---|---|---|
| SCSS-CRPS-001* | 80 | M |
| SCSS-CRPS-002 | 47 | F |
| SCSS-CRPS-003 | 42 | F |
| SCSS-CRPS-004 | 56 | F |
| SCSS-CRPS-005 | 61 | M |
| SCSS-CRPS-006 | 71 | F |
| SCSS-CRPS-007 | 63 | F |
| SCSS-CRPS-008 | 48 | F |
| SCSS-CRPS-009 | 40 | M |
| SCSS-CRPS-010 | 50 | F |

*Patient lost to follow-up

Inclusion Criteria Enrolled subjects had to have primary complaints of chronic intractable pain of the upper or lower extremity secondary to complex regional pain syndrome. Other possible etiologies of pain had to be ruled out first prior to being enrolled in the study. Subjects had to have a minimum VAS score greater than 6 in the greatest area of pain and failed to achieve adequate pain relief from at least two prior pharmacologic or interventional treatments. Subjects were greater than 18 years of age. The subjects had stable neurologic function in the past 30 days. In the investigator's opinion the subject had to be psychologically appropriate. Subjects were able to provide written and informed consent and were agreeable to the follow-up schedule and protocol. Worker's compensation enrollees had to be of Permanent and Stationary status.

Exclusion Criteria A subject is excluded if the subject has exhibited escalating or changing pain conditions in the past 30 days. Subjects currently involved in medical litigation were excluded. Subjects having had corticosteroid therapy in the past 30 days were excluded. A subject was excluded if the subject's pain medication dosage is not stable for 30 days. Subjects with cognitive, physical, or sensory impairment that in the opinion of the investigator may limit their reporting capabilities were excluded. Subjects that had active systemic infection were excluded. If the subject had significant medical comorbidity, in the opinion of the investigator, the subject was excluded. The subject was excluded if the subject participated in another clinical investigation in the past 30 days. A subject was excluded if diagnosed with cancer in the past two years. A subject was excluded if the she subject was pregnant, nursing or plans to become pregnant. Worker's Compensation subjects who have not reached maximum medical benefit were excluded from the study.

### Results

Patient demographics: Seven of the 10 subjects were female, consistent with the 3-fold higher incidence in women compared to men (Table 1). The age range of subjects was 22-72 years old. The average age was 55.8 years, also consistent with the prevalence of CRPS in older individuals One subject was lost to follow up.

Primary endpoint: Of the 10 subjects enrolled in the study, none reported any serious adverse events (SAEs) related to treatment or investigational product. Two subjects contracted COVID-19 during the study but remained enrolled and fully participated. One subject developed congestive heart failure, although this was ascribed to pre-existing comorbidities rather than the treatment protocol or investigational product.

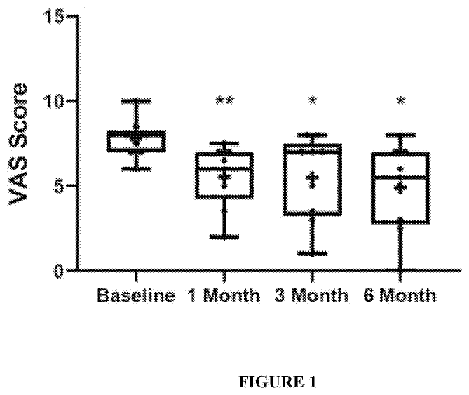

Secondary endpoints: At baseline, the average VAS scored in the 9 subjects analyzed was 7.78 (FIG. 1). The average VAS was 5.56, 5.5 and 4.89 at 1, 3, and 6 months respectively. As a group, statistically significant improvements were observed at all follow up time points. At the 1-month follow-up, one subject reported ≥50% VAS reduction. At the 3-month follow-up, three subjects reported ≥50% VAS reduction. Amongst the five subjects remaining, 4 patients reported ≥50% VAS reduction at the 6-month mark. The remaining subject reported improvements in VAS to a lesser degree.

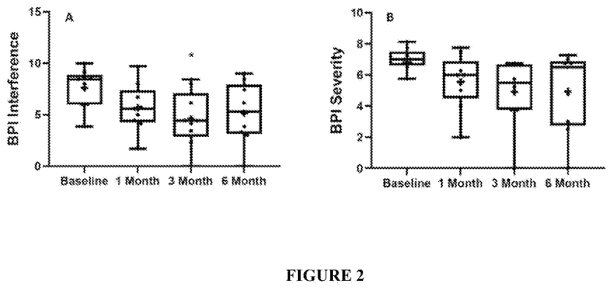

BPI pain scores were separated into Pain Interference and Pain Severity scores (FIG. 2). Trends toward improvement were observed during the study, but statistical significance was only apparent for Pain Interference at the 3-month point. Average baseline values for both were 6.99 and 7.61 for severity and interference, respectively. There were further reductions to 5.55 and 5.66, 4.90 and 4.59, and 4.94 and 5.16 at 1, 3 and 6 months when compared to baseline. There was a decrease of 29.23% and 32.22% in pain with respect to severity and interference at 6 months.

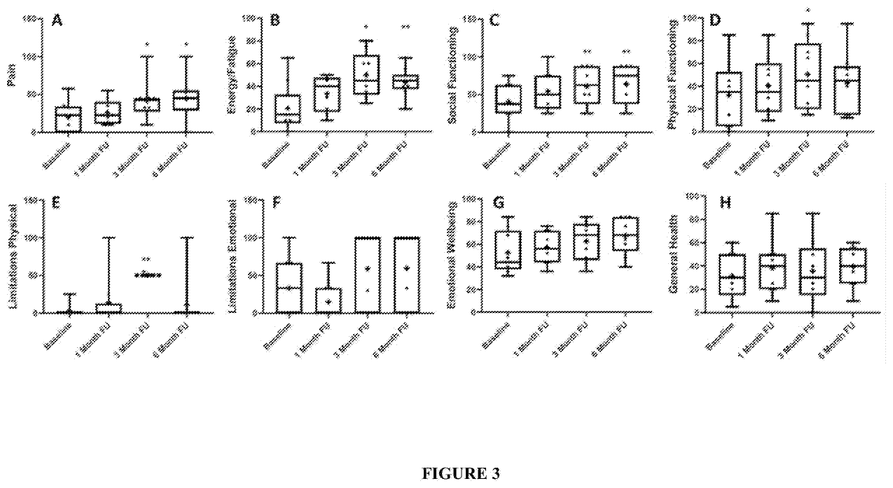

When calculating SF-36 scores, significant improvements were observed at the 3- and 6-month follow ups for pain, energy/fatigue, and social functioning metrics (FIG. 3). Significant improvements were apparent at the 3-month point for physical functioning and limitations physical categories. Trends for improvement were seen for limitations emotional and emotional wellbeing during the study but these did not reach significance. Little change was observed for the general health category.

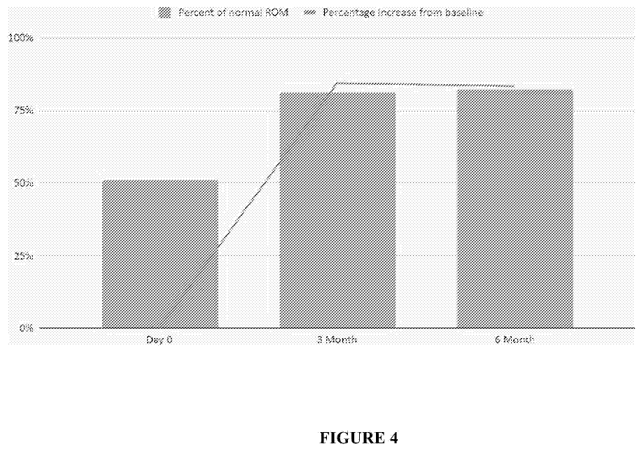

Range of motion evaluation was dependent on anatomic location of the affected joint. Data points were collected at baseline, the 3- and 6-month marks (FIG. 4). Values were recorded in degrees and then compared to normal rotational degrees of a normal joint. Of the nine subjects in the population, seven had impaired range of motion in their CRPS affected limb when compared to normal. On average, subjects had an 83.37% increase in their range of motion of their affected limb and had 82% of their normal range of motion at the end of the study (Table 2).

TABLE 2
Range of motion assessment results over the 6-month study period

| Range of motion average | Day 0 | 3 Month | 6 Month |
|---|---|---|---|
| Percent of normal ROM | 51% | 81% | 82% |
| Percentage Increase from baseline | 0% | 84.41% | 83.37% |

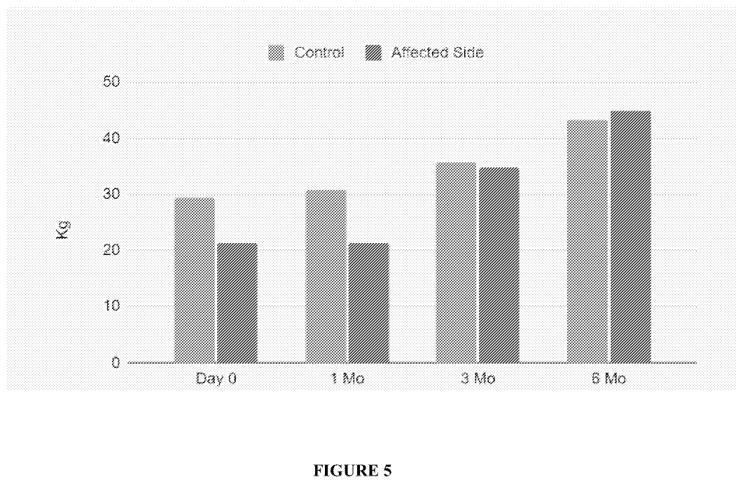

Of the nine subjects in the study, only three had grip strength affected in the upper extremity (Jamar test). Overall, grip strength improved with respect to the CRPS affected side, as well as their unaffected control side (FIG. 5). All subjects evaluated had equalization of grip strength after 6 months (Table 3).

TABLE 3
Jamar dynamometer test assessment results
over the 6-month study period

| Average (kg) | Day 0 | 1 Mo | 3 Mo | 6 Mo |
|---|---|---|---|---|
| Control | 29.33 | 30.67 | 35.67 | 43.33 |
| Affected Side | 21.33 | 21.33 | 34.67 | 45 |

### Discussion

This pilot study sought to assess the safety of ExoFlo™ investigational product in CRPS patients as a primary endpoint, and, as a secondary goal, to explore its potential for fostering improvements in symptoms associated with CRPS. The primary endpoint was met with no reported SAEs throughout the entire 6-month follow-up period. During this study, two subjects contracted COVID-19 and reported adverse complications regarding their CRPS diagnosis. One subject contracted COVID-19 between the 3- and 6-month mark, and had extremity pain, color change, and swelling return to baseline. Another subject contracted COVID-19 between the 1- and 3-month mark, and stated their neurogenic pain flare-ups had resumed, but overall had a decrease in swelling, and burning sensations as well as increased energy levels. At the six-month mark the same subject reported that burning pain was completely gone. One of the patients who contracted COVID-19 during the study, reported no change in pain interference or pain severity on the BPI scale. Between the 1- and 3-month mark, one subject was diagnosed with congestive heart failure, although, it is believed this patient did not develop congestive heart failure as a result of investigational product treatment, rather their prior comorbidities and lifestyle habits. The same subject reported a 50% decrease in pain, and overall had an increase in range of motion.

Functional assessments uniformly showed positive patient responses to the treatment. Comparison of VAS scores from baseline to 6 months showed, on average, all subjects had an improvement in pain. Overall, there was almost a 50% decrease in pain by the end of the study. One subject reported no pain at the end of the study, when at baseline their pain was rated at a 7/10. One subject reported no change in pain at baseline to 6 months but had a decrease in pain at the 1-month and 3-month follow up.

When evaluating BPI scores, on average all subjects had a decrease in pain severity and pain interference. The greatest Improvement was seen with regards to pain interference in daily life, with an overall 32% decrease in pain interference by the end of the study. One subject reported no pain interference or severity at 3 months and at the end of the 6-month trial. All but two subjects reported a decrease in pain severity throughout the trial, and the remaining two subjects reported no change. All but one subject reported a decrease in pain interference at the end of the trial, and their pain levels did not increase, but remained the same. The same subject reported no change in pain severity or pain interference when compared to baseline. This same subject contracted COVID-19 during the 1- and 3-month mark of the study. Overall, most of the observed benefit was reported by the three-month mark, with a slight rebound in pain interference at the conclusion of the 6-month trial.

Evaluation of SF-36 scores showed all subjects showed improvement at the end of the 6-month clinical trial when compared to original baseline scores. The most significant improvements were seen in pain, energy levels, social functioning and physical functioning, with a positive trend in emotional wellbeing, and the least significant improvements were seen in general health. Out of the nine subjects in this study, five reported zero emotional limitations by the end of the six-month study. One subject reported zero physical limitations and zero limitations with regards to pain by the 6-month mark, as well.

On average, the percent of normal range of motion in the affected CRPS joint was 51% when compared to the normal anatomical degrees of rotation of an unafflicted limb. By the three-month mark, the percent of normal range of motion was 81%, with an increase from baseline of 84.41%. At the six-month mark percent of normal range of motion increased to 82%, with a total increase of 83.37% from baseline. Two subjects had complete restoration of normal range of motion in their CRPS affected joint at the 6-month mark. Most of the observed benefit in range of motion was observed by the 3-month mark. On average, all patients had an increase in range of motion at 6 months when compared to baseline. There was one subject who received no benefit from therapy with regards to range of motion in one movement, extension of their shoulder, but had an increase in range of motion of flexion, as well as abduction with pronation of the same joint. No subjects reported a decrease in range of motion throughout the study.

At baseline the average grip strength of the unaffected control limb was measured at 29.33 kg, while the CRPS affected limb was 21.33 kg. At the 1-month mark, there was an increase to 30.66 kg and 21.33 kg with respect to control and CRPS afflicted side. At the 3-month mark, there was an increase to 35.67 kg and 34.67 kg, respectively. At the 6-month mark, there was an increase to 43.33 kg and 45 kg, respectively. Overall, grip strength increased by 147.73% of the control limb, and increased 210.94% of the affected CRPS limb. All subjects had an increase in grip strength on their control side, as well as their CRPS affected side. One subject had their CRPS affected side become stronger than their control side at 6 months. These subjective and objective findings were consistent with reported improvements in limb and joint range of motion, and decreased sudomotor changes, edema, allodynia, and hypesthesia.

Two subjects reported little to no hair growth on their CRPS affected limb prior to the study. Both subjects had an increase in hair growth during the study. One subject reported that by the end of the study, hair growth on the affected limb had returned to normal when compared to unaffected limb. Of the two subjects with reported hair growth deficits, one also had involvement of their nails. At the 6-month mark they reported an overall increase in nail growth rate when compared to before the study began.

Additionally, one subject had their CRPS symptoms and pain completely resolved. They reported normalization of range of motion, complete resolution of pain, no pain interference in daily life, and no pain interference with physical functioning, physical limitations, emotional limitations, decrease in energy, emotional well-being, social functioning, and general health.

### CONCLUSION

The investigational product appeared safe and well tolerated in patients with CRPS. On average, all subjects made improvements following delivery of the investigational product.

1. A method of treating complex regional pain syndrome (CRPS) in a subject in need thereof, the method comprising administering to the subject a composition comprising a therapeutic mesenchymal stem cell (MSC) secretome composition comprising extracellular vesicles, wherein at least 80% of the extracellular vesicles in the therapeutic MSC secretome composition are CD63⁺ CD9⁻ CD81⁻.

2. The method of claim 1, wherein the subject has a decrease of at least 50% in a Visual Analog Scale of Pain (VAS) score after administration compared to before administration.

3. A method of treating complex regional pain syndrome (CRPS) in a subject in need thereof, the method comprising administering to the subject a composition comprising a therapeutic mesenchymal stem cell (MSC) secretome composition comprising extracellular vesicles, wherein the subject has a decrease of at least 50% in a Visual Analog Scale of Pain (VAS) score after administration compared to before administration.

4. The method of claim 3, wherein at least 80% of the extracellular vesicles in the therapeutic MSC secretome composition are CD63⁺ CD9⁻ CD81⁻.

5. The method of any one of claims 2 to 4, wherein the subject has a minimum VAS score of greater than 6 in an affected extremity prior to administration.

6. The method of any one of claims 1 to 5, wherein the subject has a decrease in a Brief Pain Inventory (BPI) score after administration compared to before administration.

7. The method of claim 6, wherein the subject has a decrease in a BPI score of at least 10%, at least 20%, at least 30%, or greater than 30% after administration compared to before administration.

8. The method of claim 6 or 7, wherein the subject has a decrease in a BPI pain interference score of at least 10%, at least 20%, at least 30%, or greater than 30% after administration compared to before administration.

9. The method of any one of claims 1 to 8, wherein the subject has an increase in a range of motion assessment score of at least 50%, at least 60%, at least 70%, at least 75%, at least 80%, at least 85%, at least 95%, or at least 100% after administration compared to before administration.

10. The method of any one of claims 1 to 9, wherein the subject has an increase in a Jamar dynamometer test assessment score after administration as compared to before administration.

11. The method of claim 10, wherein the increase in the Jamar dynamometer test assessment score is in an upper extremity.

12. The method of claim 10 or 11, wherein the subject has an equalization of grip strength after administration.

13. The method of any one of claims 10 to 12, wherein the subject has an increase in grip strength of at least 5 kg, at least 10 kg, at least 15 kg, at or least 20 kg after administration compared to before administration.

14. The method of any one of claims 10 to 13, wherein the subject has an increase in grip strength of at least 50%, at least 75%, at least 100%, at least 125%, at least 150%, at least 175%, at least 200%, or at least 210% after administration compared to before administration.

15. The method of any one of claims 10 to 14, wherein the subject has an increase in grip strength in an affected limb.

16. The method of any one of claims 10 to 15, wherein the subject has an increase in grip strength in a control limb.

17. The method of any one of claims 10 to 16, wherein the subject has an improvement after administration compared to before administration in one or more of: limb range of motion, joint range of motion, sudomotor changes, edema, allodynia, and hypesthesia.

18. The method of any one of claims 1 to 17, wherein the subject has an improvement in an Short Form 36 Health Survey Questionnaire (SF-36) Questionnaire score after administration compared to before administration.

19. The method of claim 18, wherein the improvement in the SF-36 Questionnaire score comprises one or more of: an improvement in pain, an improvement in energy levels, an improvement in social functioning, an improvement in physical functioning, a positive trend in emotional wellbeing, and an improvement in general health.

20. The method of any one of claims 1 to 19, wherein the therapeutic MSC secretome composition further comprises one or more of the following proteins: Ferritin, NUP85, LAMP2, GPR115, Serpin F1, OPN, PAI-1, DAPP1, Cathepsin B, Semaphorin 6C, PDGF R alpha, Sortilin, Serpin B6, Dkk-3, Thrombomodulin, PF4, MIF, Periostin, Furin, TIMP-1, Decorin, PCK1, CD99, CD63, CD9, CD81, Transferrin, DcR3, Lumican, TIMP-2, SLITRK5, FAP, Artemin, DPPII, cIAP-1, Pentraxin 3, Visfatin, Neprilysin, Albumin, Galectin-1, UNC5H3, IL-20 R beta, SREC-II, JAM-C, TNF RI, htPAPP-A, eNOS, MSP R, TPP1, LAMP1, B2M, NCAM-1, HIF-1 alpha, ST6GAL1, CD99-L2, Plexin A4, EMMPRIN, p53, Semaphorin 7A, NKp80, Cystatin B, Osteoadherin, Midkine, Calreticulin, Osteoactivin, Legumain, TAZ, Cathepsin L, RBP4, Serpin A4, JAM-A, MCSF, LIMPII, OPG, IL-22, Galectin-3, MOG, Trypsin 3, SIRP alpha, and Syndecan-4, and at least one protein selected from the following: Ferritin, IGFBP-4 IL-1 R6 GSTM1, NUP85, LAMP2, MeprinA, IL-1 F10, bIG-H3, GPR115, TGFb1, Ephrin-A4, CD109, Serpin F1, IGFBP-6, HS3ST4, Aminopeptidase LRAP, OPN, PAI-1, DAPP1, GDF-9, Cathepsin B, IGFBP-2, Semaphorin 6C, IGF-2, PDGF R alpha, Sortilin, Serpin B6, Dkk-3, CNTF, TSP-1, GM-CSF Ra, Thrombomodulin, Endoglycan, IGFBP-3, RGM-C, PF4, MIF, TGM4, Periostin, Furin, TIMP-1, PAPP-A, Decorin, PCK1, Arylsulfatase A, CD99, CA2, PRDX4, Transferrin, DcR3, GP73, LAIR2, ULBP-4, Lumican, TIMP-2, TFPI, SOX2, SLITRK5, FAP, Spinesin, ENPP-2, CD97, CTACK, Integrin alpha 1, EXTL3, IL-18 BPa, PD-L2, PSMA, IL-20 Ra, Glyoxalase II, Trypsin 1, IGF-2R, ADAMTSL-1, Erythropoietin, Plexin D1, DNMT3A, BCL-2, CL-P1, Ephrin-B3, FABP6, CHI3L1, FCRLS, TFF3, Artemin, DPPII, cIAP-1, PDGF Rb, Pentraxin 3, Angiotensinogen, Follistatin, CF VII, Persephin, TRAIL R1, THAP11, CD200, CLEC-2, AMIGO, IGFBP-5, PON1, SOX7, GALNT10, Visfatin, Progranulin, PCSK2, GKN1, IL-18, Neprilysin, Stabilin-2, IL-17 RD, Albumin, Follistatin-like 1, MMP-10, FKBP51, LRRC4, Pref-1, Galectin-1, Troponin C, UNC5H3, FLRT2, CD314, Semaphorin 6B, Netrin-4, CD27 Ligand, IL-20 R beta, Semaphorin 6A, TSK, Cytokeratin-8, CHST3, Mcl-1, DPPIV, SREC-IL, Norrin, JAM-C, Bcl-10, Wnt-4, LSECtin, Kell, TNF RI, PTP1B, htPAPP-A, IDO, PDGF-CC, Galanin, Activin A, TLR2, SCCA2, FABP1, eNOS, SHP-1, ICOS, ClqTNF9, MMP-1, TC-PTP, IL-24, gp130, C-myc, LILRB4, BMP-2, MIA, CD34, CD63, CD9, CD81, IFNab R2, Glypican 2, MSP R, DSCAM, Matriptase, KIR2DL3, CD30, Siglec-10, CLEC-1, TPP1, Ubiquitin+1, ANGPTL4, TWEAK R, Nidogen-1, CD2, Kallikrein 1, TSLP R, LAMP1, TROY, VCAM-1, Siglec-11, S100A1, PAR1, Thyroid Peroxidase, Aminopeptidase P2, IL-1 RI, ADAMS, OSM R beta, Thrombospondin-2, SMPD1, B2M, MFRP, LRP-6, ST3GAL1, NCAM-1 (CD56), Granzyme B, Adiponectin, IL-22BP, TPST2, PD-ECGF, LH, LEDGF, Cyr61, ULBP-3, IFNb, THSD1, FGF-23, LAMA4, Adipsin, AIF, SorCS2, SULT2A1, CD39L2, Insulin R, HIF-1 alpha, OX40 Ligand, Pax3, UCH-L3, cMASP3, Langerin, Desmin, SOX9, ST6GAL1, MEP1B, CD99-L2, Plexin A4, Semaphorin 4D, ROBO2, PDX-1, APRIL, Neurturin, Kremen-2, EMMPRIN, Activin RIB, Neuroligin 2, Epiregulin, CASA, MMP-12, GALNT2, CEACAM-5, VEGF R1, DSPG3, SorCS1, Matrilin-2, sFRP-3, p53, EphB3, NCK1, Semaphorin 7A, NKp80, Prolactin, Cystatin B, Sirtuin 1, FGF-16, FGF R5, NQO-1, Semaphorin 6D, FGF-3, GATA-4, VAP-A, CHST2, Pappalysin-2, Syndecan-3, Jagged 1, AKR1C4, Olfactomedin-2, Osteoadherin, NKp44, Thyroglobulin, IL-21R, Chemerin, EphA1, CD48, MICB, FGF-5, TRANCE, CES2, ULBP-1, Integrin alpha 5, VAMP-2, FLRG, Ret Midkine, CD73, TRACP, proGRP, Granzyme H, PRX2, p2′7, Siglec-6, Dectin-1, CD51, Notch-1, Calreticulin, DR3, DCTN1, CDC25B, Osteoactivin, ACE, CA125, HAO-1, PSMA1, FCRLB, BMP-9, CRIM1, LIF, SPINK1, EphB6, RGM-B, HS3ST1, ROR1, CMG-2, 4-1BB Ligand, L1CAM-2, p63, Cathepsin V, Testican 2, Glypican 5, CD6, Siglec-2, Legumain, PRELP, CES1, TAZ, NSE, TECK, HTRA2, HIF-1 beta, TAFA1, Podocalyxin, RalA, CRELD2, GRAP2, SP-D, BID, GFR alpha-2, Notch-3, VEGF R3, DLL4, TGFb2, LIGHT, XIAP, ST8SIA1, Cathepsin L, 6Ckine, MIS RII, Kallikrein 5, TGM3, FCAR, Contactin-2, CD83, IL-1 R3, SALM4, GBA3, ROBO4, OSCAR, VEGF, IGSF3, Biglycan, Neudesin, ILT4, uPAR, Axl, WIF-1, IL-7 R alpha, GPR56, CEACAM-3, MCEMP1, FABP2, Plexin B3, MEPE, Activin RIIA, ANG-2, Cochlin, Presenilin 1, NPTXR, SLAM, COMT, SPHK1, RBP4, Nectin-1, GUSB, Nidogen-2, IL-17F, SR-AI, TAFA2, N-Cadherin, IL-17B, IL-17 RC, MIP-3b, Cystatin C, Cystatin D, AMSH, FcERI, CLEC10A, HGF R, ANG-1, Prolactin R, FGF-20, CD28, Nogo-A, HSD17B1, IL-19, Enteropeptidase, Cathepsin E, TSLP, TCN2, GDF-15, Epimorphin, GRKS, PD-1, Serpin A4, ADAM23, NOV, Galectin-2, Neurexin 3 beta, TLR3, Sirtuin 2, Numb, IL-28 R alpha, IL-33, Lin28, FCRL1, KLF4, NKp30, Lymphotactin, Cystatin SN, JAM-A, Calreticulin-2, ErbB4, BMP-8, IL-27 Ra, Fas, IL-4 Ra, Kallikrein 14, Matrilin-3, Olig2, Kallikrein 12, CA13, IL-9, Nectin-3, MPIF-1, Cystatin S, ADA, IL-2 Rb, GFR alpha-1, Smad4, ICAM-1, MEF2C, TREM-1, L-Selectin, Hepsin, CD42b, MCSF, RANK, CHST4, CA8, FCRL3, ASAH2, CF XIV, PYY, HGF, I-TAC, Semaphorin 4C, SorCS3, Tie-1, IL-31 RA, Arginase 1, POGLUT1, IL-Ira, Podoplanin, TIM-3, CREG, CD300f, uPA, EphA2, LRRTM4, LIMPII, Tenascin R, CPE, PECAM-1, DNAM-1, DKK-1, OPG, CPB1, TSH, MMP-2, Siglec-9, ICAM-3, Cystatin SA, Galectin-4, Pepsinogen II, Desmoglein-3, Nectin-4, SCF, Serpin A5, PTH, FGF-19, MSP, IL-28A, FGF-12, METAP2, ASAHL, EDIL3, NTAL, EGF R, TAFAS, Galectin-9, vWF-A2, TACE, Activin RIM, Cathepsin S, LDL R, BMPR-IA, OX40, IL-13 R2, B7-H4, MMP-13, ANGPTL7, TRAIL R4, IGSF4B, Sirtuin 5, PEAR1, SH2D1A, Cerberus 1, GDF-11, Nrf2, TROP-2, NUDTS, ROR2, EphB4, Glypican 1, LAP(TGFb1), Gash, Contactin-1, IL-27, UNC5H4, ICAM-2, MBL, HS3ST3B1, RCOR1, IL-10 Rb, XEDAR, IL-22, PILR-alpha, NRG1-131, FABP4, RGM-A, RELT, TrkC, CSa, SREC-I, Nestin, TPO, ErbB3, Kirre13, FLRT1, Galectin-3, CXCL16, JAM-B, DR6, Nogo Receptor, TLR4, VEGF R2, Tie-2, IL-15 R, Caspr2, LTbR, LAMP, ALCAM, GLP-1, NG2, IL-22 R alpha 1, AMIGO2, HCC-1, TFPI-2, ULBP-2, Desmoglein 2, Aggrecan, Syntaxin 4, VAMP-1, Nectin-2, FGF-21, Flt-3, GFAP, TIM-1, Inhibin A, Cadherin-4, P1GF-2, Neurogranin, HE4, IL-23 R, Galectin-7, GALNT3, GITR L, CD14, R-Spondin 2, CK19, Cardiotrophin-1, TREML1, HAPLN1, CD27, ANG-4, Siglec-7, CD155, VEGF-C, TNF RII, PGRP-S, SDF-1a, PDGF-AB, GPVI, CD40, SCF R, Thrombospondin-5, IL-1 RII, Neuropilin-2, Cadherin-13, E-Selectin, GITR, WISP-1, Renin, AgRP, MDL-1, ROBO3, RANTES, Endocan, Granulysin, hCGb, Mesothelin, TLR1, TRAIL, MOG, DDR1, NGF R, TRAIL R3, Trypsin 3, ARSB, LIF R alpha, BAFF R, CD157, Granzyme A, 2B4, ESAM, IL-1 R4, CXCL14, IL-31, SIRP alpha, Uromodulin, CTRC, CEACAM-1, TARC, MIP-3a, SDF-1b, NKp46, MCP-3, IL-32 alpha, TGFb3 FOLR2, CD58, IL-23, CD36, TNFb, Shh-N, Ficolin-1, Reg4, ILT2, Mer, TREM-2, Flt-3L, CDS, IL-6, CD229, Insulin, Syntaxin 6, GRO, Bcl-w, Lipocalin-2, PDGF-AA, IL-2 Ra, Angiogenin, LYVE-1, CD4, RAGE, CDNF, Brevican, NAP-2, PU.1, EDAR, ADAMTS13, Kynureninase, PTH1R, IFN-gamma R1, CrkL, B7-1, PARC, Draxin, VE-Cadherin, Procalcitonin, SOX15, Kallikrein 11, BCMA, Dectin-2, EpCAM, HCC-4, TGFa, IP-10, BLAME, CILP-1, PIGF, LOX-1, MCP-2, Resistin, HVEM, ENPP-7, Syndecan-4, IL-2 Rg, MICA, Dopa Decarboxylase, NPDC-1, MCP-4, EG-VEGF, Glycoprotein V, Semaphorin 4G, IL-12p40, PSA-total, IL-15, MAP1D, C1q, TNF4, Dtk, Endoglin, ENA-78, Reg3A, MIP-1b, FGF-17, IL-6R, IL-8, Galectin-8, CA4, Cystatin E M, FUT8, B7-H3, GCP-2, CD40L, MDC, 4-1BB, HO-1, SOST, S100A13, Kallikrein 7, or IL-13.

21. The method of any one of claims 1 to 20, wherein the extracellular vesicles comprise one or more of the following nucleic acids: hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-1′7-5p, hsa-miR-181a-5p, hsa-miR-191-5p, hsa-miR-193a-5p, hsa-miR-193b-3p, hsa-miR-19′7-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, or hsa-miR-99b-5p.

22. The method of any one of claims 1 to 21, wherein the subject has chronic intractable pain of one or more upper or lower extremities.

23. The method of any one of claims 1 to 22, wherein the subject does not experience escalating or changing pain in the 30 days preceding administration.

24. The method of any one of claims 1 to 23, wherein the subject experiences decreased pain flare-ups, decreased swelling, and/or decreased burning sensations in an affected limb after administration compared to before administration.

25. The method of any one of claims 1 to 24, wherein the administration improves hair and/or nail growth in an affected limb of the subject.

26. The method of any one of claims 1 to 25, wherein the composition is produced by:
(a) culturing bone marrow-derived MSCs under the following conditions to produce an MSC conditioned media:
(i) oxygen tension below 5%; and
(ii) culture media having a pH below 7;
(b) harvesting the MSC conditioned media; and
(c) formulating the MSC conditioned media to produce the therapeutic MSC secretome composition, wherein the therapeutic MSC secretome composition comprises proteins and extracellular vesicles produced by the bone marrow-derived MSCs in step (a).

27. The method of claim 26, wherein the culture media is serum-free.

28. The method of claim 26 or 27, wherein the culture media has a glucose concentration below 4.5 g/L.

29. The method of any one of claims 1 to 28, wherein the subject is human.

30. The method of any one of claims 26 to 29, wherein the bone-marrow derived MSCs are derived from human bone marrow.

31. The method of any one of claims 1 to 30, wherein administering comprises intravenous administration.

32. The method of any one of claims 1 to 31, wherein the dosage of the therapeutic MSC secretome composition administered to the subject is a cell-equivalent dosage of 0.7 to 7 million cells/kg.

33. The method of any one of claims 1 to 31, wherein the therapeutic MSC secretome composition comprises 4×10¹⁰ to 10×10¹⁰ cells/mL.

34. The method of any one of claims 1 to 31, wherein the therapeutic MSC secretome composition comprises 5×10¹¹ to 1.5×10¹² extracellular vesicles.

35. The method of any one of claims 1 to 34, wherein the composition is administered monthly for two or more months, or once every 1, 2, or 3 or more months.

36. The method of any one of claims 1 to 34, wherein the composition is administered twice.

37. The method of claim 36, wherein the composition is administered 4 days apart.

38. A method of making a composition comprising a therapeutic mesenchymal stem cell (MSC) secretome composition for treating complex regional pain syndrome (CRPS) in a subject in need thereof, the method comprising:
(a) culturing bone marrow-derived MSCs under the following conditions to produce an MSC conditioned media:
(i) oxygen tension below 5%; and
(ii) culture media having a pH below 7;
(b) harvesting the MSC conditioned media; and
(c) formulating the MSC conditioned media to produce the therapeutic MSC secretome composition, wherein the therapeutic MSC secretome composition comprises proteins and extracellular vesicles produced by the bone marrow-derived MSCs in step (a).

39. The method of claim 38, wherein the culture media is serum-free.

40. The method of claim 38 or 39, wherein the culture media has a glucose concentration below 4.5 g/L.

41. The method of any one of claims 38 to 40, wherein at least 80% of the extracellular vesicles in the therapeutic MSC secretome composition are CD63⁺ CD9⁻ CD81⁻.

42. The method of any one of claims 38 to 41, wherein the bone-marrow derived MSCs are derived from human bone marrow.

43. The method of any one of claims 38 to 42, wherein administering comprises intravenous administration.

44. The method of any one of claims 38 to 43, wherein the therapeutic MSC secretome composition further comprises one or more of the following proteins: Ferritin, NUP85, LAMP2, GPR115, Serpin F1, OPN, PAI-1, DAPP1, Cathepsin B, Semaphorin 6C, PDGF R alpha, Sortilin, Serpin B6, Dkk-3, Thrombomodulin, PF4, MIF, Periostin, Furin, TIMP-1, Decorin, PCK1, CD99, CD63, CD9, CD81, Transferrin, DcR3, Lumican, TIMP-2, SLITRK5, FAP, Artemin, DPPII, cIAP-1, Pentraxin 3, Visfatin, Neprilysin, Albumin, Galectin-1, UNC5H3, IL-20 R beta, SREC-II, JAM-C, TNF RI, htPAPP-A, eNOS, MSP R, TPP1, LAMP1, B2M, NCAM-1, HIF-1 alpha, ST6GAL1, CD99-L2, Plexin A4, EMMPRIN, p53, Semaphorin 7A, NKp80, Cystatin B, Osteoadherin, Midkine, Calreticulin, Osteoactivin, Legumain, TAZ, Cathepsin L, RBP4, Serpin A4, JAM-A, MCSF, LIMPII, OPG, IL-22, Galectin-3, MOG, Trypsin 3, SIRP alpha, and Syndecan-4, and at least one protein selected from the following: Ferritin, IGFBP-4 IL-1 R6 GSTM1, NUP85, LAMP2, MeprinA, IL-1 F10, bIG-H3, GPR115, TGFb1, Ephrin-A4, CD109, Serpin F1, IGFBP-6, HS3ST4, Aminopeptidase LRAP, OPN, PAI-1, DAPP1, GDF-9, Cathepsin B, IGFBP-2, Semaphorin 6C, IGF-2, PDGF R alpha, Sortilin, Serpin B6, Dkk-3, CNTF, TSP-1, GM-CSF Ra, Thrombomodulin, Endoglycan, IGFBP-3, RGM-C, PF4, MIF, TGM4, Periostin, Furin, TIMP-1, PAPP-A, Decorin, PCK1, Arylsulfatase A, CD99, CA2, PRDX4, Transferrin, DcR3, GP73, LAIR2, ULBP-4, Lumican, TIMP-2, TFPI, SOX2, SLITRK5, FAP, Spinesin, ENPP-2, CD97, CTACK, Integrin alpha 1, EXTL3, IL-18 BPa, PD-L2, PSMA, IL-20 Ra, Glyoxalase II, Trypsin 1, IGF-2R, ADAMTSL-1, Erythropoietin, Plexin D1, DNMT3A, BCL-2, CL-P1, Ephrin-B3, FABP6, CHI3L1, FCRLS, TFF3, Artemin, DPPII, cIAP-1, PDGF Rb, Pentraxin 3, Angiotensinogen, Follistatin, CF VII, Persephin, TRAIL R1, THAP11, CD200, CLEC-2, AMIGO, IGFBP-5, PON1, SOX7, GALNT10, Visfatin, Progranulin, PCSK2, GKN1, IL-18, Neprilysin, Stabilin-2, IL-17 RD, Albumin, Follistatin-like 1, MMP-10, FKBP51, LRRC4, Pref-1, Galectin-1, Troponin C, UNC5H3, FLRT2, CD314, Semaphorin 6B, Netrin-4, CD27 Ligand, IL-20 R beta, Semaphorin 6A, TSK, Cytokeratin-8, CHST3, Mcl-1, DPPIV, SREC-IL, Norrin, JAM-C, Bcl-10, Wnt-4, LSECtin, Kell, TNF RI, PTP1B, htPAPP-A, IDO, PDGF-CC, Galanin, Activin A, TLR2, SCCA2, FABP1, eNOS, SHP-1, ICOS, ClqTNF9, MMP-1, TC-PTP, IL-24, gp130, C-myc, LILRB4, BMP-2, MIA, CD34, CD63, CD9, CD81, IFNab R2, Glypican 2, MSP R, DSCAM, Matriptase, KIR2DL3, CD30, Siglec-10, CLEC-1, TPP1, Ubiquitin+1, ANGPTL4, TWEAK R, Nidogen-1, CD2, Kallikrein 1, TSLP R, LAMP1, TROY, VCAM-1, Siglec-11, S100A1, PAR1, Thyroid Peroxidase, Aminopeptidase P2, IL-1 RI, ADAMS, OSM R beta, Thrombospondin-2, SMPD1, B2M, MFRP, LRP-6, ST3GAL1, NCAM-1 (CD56), Granzyme B, Adiponectin, IL-22BP, TPST2, PD-ECGF, LH, LEDGF, Cyr61, ULBP-3, IFNb, THSD1, FGF-23, LAMA4, Adipsin, AIF, SorCS2, SULT2A1, CD39L2, Insulin R, HIF-1 alpha, OX40 Ligand, Pax3, UCH-L3, cMASP3, Langerin, Desmin, SOX9, ST6GAL1, MEP1B, CD99-L2, Plexin A4, Semaphorin 4D, ROBO2, PDX-1, APRIL, Neurturin, Kremen-2, EMMPRIN, Activin RIB, Neuroligin 2, Epiregulin, CASA, MMP-12, GALNT2, CEACAM-5, VEGF R1, DSPG3, SorCS1, Matrilin-2, sFRP-3, p53, EphB3, NCK1, Semaphorin 7A, NKp80, Prolactin, Cystatin B, Sirtuin 1, FGF-16, FGF R5, NQO-1, Semaphorin 6D, FGF-3, GATA-4, VAP-A, CHST2, Pappalysin-2, Syndecan-3, Jagged 1, AKR1C4, Olfactomedin-2, Osteoadherin, NKp44, Thyroglobulin, IL-21R, Chemerin, EphA1, CD48, MICB, FGF-5, TRANCE, CES2, ULBP-1, Integrin alpha 5, VAMP-2, FLRG, Ret Midkine, CD73, TRACP, proGRP, Granzyme H, PRX2, p2′7, Siglec-6, Dectin-1, CD51, Notch-1, Calreticulin, DR3, DCTN1, CDC25B, Osteoactivin, ACE, CA125, HAO-1, PSMA1, FCRLB, BMP-9, CRIM1, LIF, SPINK1, EphB6, RGM-B, HS3ST1, ROR1, CMG-2, 4-1BB Ligand, L1CAM-2, p63, Cathepsin V, Testican 2, Glypican 5, CD6, Siglec-2, Legumain, PRELP, CES1, TAZ, NSE, TECK, HTRA2, HIF-1 beta, TAFA1, Podocalyxin, RalA, CRELD2, GRAP2, SP-D, BID, GFR alpha-2, Notch-3, VEGF R3, DLL4, TGFb2, LIGHT, XIAP, ST8SIA1, Cathepsin L, 6Ckine, MIS RII, Kallikrein 5, TGM3, FCAR, Contactin-2, CD83, IL-1 R3, SALM4, GBA3, ROBO4, OSCAR, VEGF, IGSF3, Biglycan, Neudesin, ILT4, uPAR, Axl, WIF-1, IL-7 R alpha, GPR56, CEACAM-3, MCEMP1, FABP2, Plexin B3, MEPE, Activin RIIA, ANG-2, Cochlin, Presenilin 1, NPTXR, SLAM, COMT, SPHK1, RBP4, Nectin-1, GUSB, Nidogen-2, IL-17F, SR-AI, TAFA2, N-Cadherin, IL-17B, IL-17 RC, MIP-3b, Cystatin C, Cystatin D, AMSH, FcERI, CLEC10A, HGF R, ANG-1, Prolactin R, FGF-20, CD28, Nogo-A, HSD17B1, IL-19, Enteropeptidase, Cathepsin E, TSLP, TCN2, GDF-15, Epimorphin, GRKS, PD-1, Serpin A4, ADAM23, NOV, Galectin-2, Neurexin 3 beta, TLR3, Sirtuin 2, Numb, IL-28 R alpha, IL-33, Lin28, FCRL1, KLF4, NKp30, Lymphotactin, Cystatin SN, JAM-A, Calreticulin-2, ErbB4, BMP-8, IL-27 Ra, Fas, IL-4 Ra, Kallikrein 14, Matrilin-3, Olig2, Kallikrein 12, CA13, IL-9, Nectin-3, MPIF-1, Cystatin S, ADA, IL-2 Rb, GFR alpha-1, Smad4, ICAM-1, MEF2C, TREM-1, L-Selectin, Hepsin, CD42b, MCSF, RANK, CHST4, CA8, FCRL3, ASAH2, CF XIV, PYY, HGF, I-TAC, Semaphorin 4C, SorCS3, Tie-1, IL-31 RA, Arginase 1, POGLUT1, IL-Ira, Podoplanin, TIM-3, CREG, CD300f, uPA, EphA2, LRRTM4, LIMPII, Tenascin R, CPE, PECAM-1, DNAM-1, DKK-1, OPG, CPB1, TSH, MMP-2, Siglec-9, ICAM-3, Cystatin SA, Galectin-4, Pepsinogen II, Desmoglein-3, Nectin-4, SCF, Serpin A5, PTH, FGF-19, MSP, IL-28A, FGF-12, METAP2, ASAHL, EDIL3, NTAL, EGF R, TAFAS, Galectin-9, vWF-A2, TACE, Activin RIM, Cathepsin S, LDL R, BMPR-IA, OX40, IL-13 R2, B7-H4, MMP-13, ANGPTL7, TRAIL R4, IGSF4B, Sirtuin 5, PEAR1, SH2D1A, Cerberus 1, GDF-11, Nrf2, TROP-2, NUDTS, ROR2, EphB4, Glypican 1, LAP(TGFb1), Gash, Contactin-1, IL-27, UNC5H4, ICAM-2, MBL, HS3ST3B1, RCOR1, IL-10 Rb, XEDAR, IL-22, PILR-alpha, NRG1-131, FABP4, RGM-A, RELT, TrkC, CSa, SREC-I, Nestin, TPO, ErbB3, Kirre13, FLRT1, Galectin-3, CXCL16, JAM-B, DR6, Nogo Receptor, TLR4, VEGF R2, Tie-2, IL-15 R, Caspr2, LTbR, LAMP, ALCAM, GLP-1, NG2, IL-22 R alpha 1, AMIGO2, HCC-1, TFPI-2, ULBP-2, Desmoglein 2, Aggrecan, Syntaxin 4, VAMP-1, Nectin-2, FGF-21, Flt-3, GFAP, TIM-1, Inhibin A, Cadherin-4, P1GF-2, Neurogranin, HE4, IL-23 R, Galectin-7, GALNT3, GITR L, CD14, R-Spondin 2, CK19, Cardiotrophin-1, TREML1, HAPLN1, CD27, ANG-4, Siglec-7, CD155, VEGF-C, TNF RII, PGRP-S, SDF-1a, PDGF-AB, GPVI, CD40, SCF R, Thrombospondin-5, IL-1 RII, Neuropilin-2, Cadherin-13, E-Selectin, GITR, WISP-1, Renin, AgRP, MDL-1, ROBO3, RANTES, Endocan, Granulysin, hCGb, Mesothelin, TLR1, TRAIL, MOG, DDR1, NGF R, TRAIL R3, Trypsin 3, ARSB, LIF R alpha, BAFF R, CD157, Granzyme A, 2B4, ESAM, IL-1 R4, CXCL14, IL-31, SIRP alpha, Uromodulin, CTRC, CEACAM-1, TARC, MIP-3a, SDF-1b, NKp46, MCP-3, IL-32 alpha, TGFb3 FOLR2, CD58, IL-23, CD36, TNFb, Shh-N, Ficolin-1, Reg4, ILT2, Mer, TREM-2, Flt-3L, CDS, IL-6, CD229, Insulin, Syntaxin 6, GRO, Bcl-w, Lipocalin-2, PDGF-AA, IL-2 Ra, Angiogenin, LYVE-1, CD4, RAGE, CDNF, Brevican, NAP-2, PU.1, EDAR, ADAMTS13, Kynureninase, PTH1R, IFN-gamma R1, CrkL, B7-1, PARC, Draxin, VE-Cadherin, Procalcitonin, SOX15, Kallikrein 11, BCMA, Dectin-2, EpCAM, HCC-4, TGFa, IP-10, BLAME, CILP-1, PIGF, LOX-1, MCP-2, Resistin, HVEM, ENPP-7, Syndecan-4, IL-2 Rg, MICA, Dopa Decarboxylase, NPDC-1, MCP-4, EG-VEGF, Glycoprotein V, Semaphorin 4G, IL-12p40, PSA-total, IL-15, MAP1D, C1q, TNF4, Dtk, Endoglin, ENA-78, Reg3A, MIP-1b, FGF-17, IL-6R, IL-8, Galectin-8, CA4, Cystatin E M, FUT8, B7-H3, GCP-2, CD40L, MDC, 4-1BB, HO-1, SOST, S100A13, Kallikrein 7, or IL-13.

45. The method of any one of claims 38 to 44, wherein the extracellular vesicles comprise one or more of the following nucleic acids: hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-1′7-5p, hsa-miR-181a-5p, hsa-miR-191-5p, hsa-miR-193a-5p, hsa-miR-193b-3p, hsa-miR-19′7-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, or hsa-miR-99b-5p.

46. Use of the composition produced by the method of any one of claims 38 to 45 in treating complex regional pain syndrome (CRPS) in a subject in need thereof.

47. The use of claim 46, wherein the subject is human.

48. The use of claim 46 or 47, wherein the subject has a decrease of at least 50% in a Visual Analog Scale of Pain (VAS) score after administration compared to before administration.

49. The use of any one of claims 46 to 48, wherein the subject has a minimum VAS score of greater than 6 in an affected extremity prior to administration.

50. The use of any one of claims 46 to 49, wherein the subject has a decrease in a Brief Pain Inventory (BPI) score after administration compared to before administration.

51. The use of claim 50, wherein the subject has a decrease in a BPI score of at least 10%, at least 20%, at least 30%, or greater than 30% after administration compared to before administration.

52. The use of claim 50 or 51, wherein the subject has a decrease in a BPI pain interference score of at least 10%, at least 20%, at least 30%, or greater than 30% after administration compared to before administration.

53. The use of any one of claims 46 to 52, wherein the subject has an increase in a range of motion assessment score of at least 50%, at least 60%, at least 70%, at least 75%, at least 80%, at least 85%, at least 95%, or at least 100% after administration compared to before administration.

54. The use of any one of claims 46 to 53, wherein the subject has an increase in a Jamar dynamometer test assessment score after administration as compared to before administration.

55. The use of claim 54, wherein the increase in the Jamar dynamometer test assessment score is in an upper extremity.

56. The use of claim 54 or 55, wherein the subject has an equalization of grip strength after administration.

57. The use of any one of claims 54 to 56, wherein the subject has an increase in grip strength of at least 5 kg, at least 10 kg, at least 15 kg, at or least 20 kg after administration compared to before administration.

58. The use of any one of claims 54 to 57, wherein the subject has an increase in grip strength of at least 50%, at least 75%, at least 100%, at least 125%, at least 150%, at least 175%, at least 200%, or at least 210% after administration compared to before administration.

59. The use of any one of claims 54 to 58, wherein the subject has an increase in grip strength in an affected limb.

60. The use of any one of claims 54 to 59, wherein the subject has an increase in grip strength in a control limb.

61. The use of any one of claims 46 to 60, wherein the subject has an improvement after administration compared to before administration in one or more of: limb range of motion, joint range of motion, sudomotor changes, edema, allodynia, and hypesthesia.

62. The use of any one of claims 46 to 61, wherein the subject has an improvement in an Short Form 36 Health Survey Questionnaire (SF-36) Questionnaire score after administration compared to before administration.

63. The use of claim 62, wherein the improvement in the SF-36 Questionnaire score comprises one or more of: an improvement in pain, an improvement in energy levels, an improvement in social functioning, an improvement in physical functioning, a positive trend in emotional wellbeing, and an improvement in general health.

64. The use of any one of claims 46 to 63, wherein the subject has chronic intractable pain of one or more upper or lower extremities.

65. The use of any one of claims 46 to 64, wherein the subject does not experience escalating or changing pain in the 30 days preceding administration.

66. The use of any one of claims 46 to 65, wherein the subject experiences decreased pain flare-ups, decreased swelling, and/or decreased burning sensations in an affected limb after administration compared to before administration.

67. The use of any one of claims 46 to 66, wherein the administration improves hair and/or nail growth in an affected limb of the subject.

68. The use of any one of claims 46 to 67, wherein the dosage of the therapeutic MSC secretome composition administered to the subject is a cell-equivalent dosage of 0.7 to 7 million cells/kg.

69. The method of any one of claims 46 to 67, wherein the therapeutic MSC secretome composition comprises 4×10¹⁰ to 10×10¹⁰ cells/mL.

70. The method of any one of claims 46 to 67, wherein the therapeutic MSC secretome composition comprises 5×10¹¹ to 1.5×10¹² extracellular vesicles.

71. The method of any one of claims 38 to 70, wherein the composition is administered monthly for two or more months, or once every 1, 2, or 3 or more months.

72. The method of any one of claims 38 to 70, wherein the composition is administered twice.

73. The method of claim 72, wherein the composition is administered 4 days apart.

74. Use of a composition comprising a therapeutic mesenchymal stem cell (MSC) secretome composition comprising extracellular vesicles in treating complex regional pain syndrome (CRPS) in a subject in need thereof, wherein at least 80% of the extracellular vesicles in the therapeutic MSC secretome composition are CD63+ CD9− CD81−.

75. The use of claim 74, wherein the composition is intravenously administered to the subject.

76. The use of claim 74 or 75, wherein the subject has a decrease of at least 50% in a Visual Analog Scale of Pain (VAS) score after administration of the therapeutic MSC secretome composition compared to before administration of the therapeutic MSC secretome composition.

77. The use of any one of claims 74 to 76, wherein the subject has a minimum VAS score of greater than 6 in an affected extremity prior to administration of the therapeutic MSC secretome composition.

78. The use of any one of claims 74 to 77, wherein the subject has a decrease in a Brief Pain Inventory (BPI) score after administration of the therapeutic MSC secretome composition compared to before administration of the therapeutic MSC secretome composition.

79. Use of a composition comprising a therapeutic mesenchymal stem cell (MSC) secretome composition comprising extracellular vesicles in treating complex regional pain syndrome (CRPS) in a subject in need thereof, wherein the subject has a decrease of at least 50% in a Visual Analog Scale of Pain (VAS) score after administration of the therapeutic MSC secretome composition. compared to before administration of the therapeutic MSC secretome composition.

80. The use of claim 79, wherein the subject has a minimum VAS score of greater than 6 in an affected extremity prior to administration of the therapeutic MSC secretome composition.

81. The use of claim 79 or 80, wherein the subject has a decrease in a Brief Pain Inventory (BPI) score after administration of the therapeutic MSC secretome composition compared to before administration of the therapeutic MSC secretome composition.

82. The use of any one of claims 79 to 81, wherein at least 80% of the extracellular vesicles in the therapeutic MSC secretome composition are CD63⁺ CD9⁻ CD81⁻.

83. The use of any one of claims 79 to 82, wherein the therapeutic MSC secretome composition further comprises one or more of the following proteins: Ferritin, NUP85, LAMP2, GPR115, Serpin F1, OPN, PAI-1, DAPP1, Cathepsin B, Semaphorin 6C, PDGF R alpha, Sortilin, Serpin B6, Dkk-3, Thrombomodulin, PF4, MIF, Periostin, Furin, TIMP-1, Decorin, PCK1, CD99, CD63, CD9, CD81, Transferrin, DcR3, Lumican, TIMP-2, SLITRK5, FAP, Artemin, DPPII, cIAP-1, Pentraxin 3, Visfatin, Neprilysin, Albumin, Galectin-1, UNC5H3, IL-20 R beta, SREC-II, JAM-C, TNF RI, htPAPP-A, eNOS, MSP R, TPP1, LAMP1, B2M, NCAM-1, HIF-1 alpha, ST6GAL1, CD99-L2, Plexin A4, EMMPRIN, p53, Semaphorin 7A, NKp80, Cystatin B, Osteoadherin, Midkine, Calreticulin, Osteoactivin, Legumain, TAZ, Cathepsin L, RBP4, Serpin A4, JAM-A, MCSF, LIMPII, OPG, IL-22, Galectin-3, MOG, Trypsin 3, SIRP alpha, and Syndecan-4, and at least one protein selected from the following: Ferritin, IGFBP-4 IL-1 R6 GSTM1, NUP85, LAMP2, MeprinA, IL-1 F10, bIG-H3, GPR115, TGFb1, Ephrin-A4, CD109, Serpin F1, IGFBP-6, HS3ST4, Aminopeptidase LRAP, OPN, PAI-1, DAPP1, GDF-9, Cathepsin B, IGFBP-2, Semaphorin 6C, IGF-2, PDGF R alpha, Sortilin, Serpin B6, Dkk-3, CNTF, TSP-1, GM-CSF Ra, Thrombomodulin, Endoglycan, IGFBP-3, RGM-C, PF4, MIF, TGM4, Periostin, Furin, TIMP-1, PAPP-A, Decorin, PCK1, Arylsulfatase A, CD99, CA2, PRDX4, Transferrin, DcR3, GP73, LAIR2, ULBP-4, Lumican, TIMP-2, TFPI, SOX2, SLITRK5, FAP, Spinesin, ENPP-2, CD97, CTACK, Integrin alpha 1, EXTL3, IL-18 BPa, PD-L2, PSMA, IL-20 Ra, Glyoxalase II, Trypsin 1, IGF-2R, ADAMTSL-1, Erythropoietin, Plexin D1, DNMT3A, BCL-2, CL-P1, Ephrin-B3, FABP6, CHI3L1, FCRLS, TFF3, Artemin, DPPII, cIAP-1, PDGF Rb, Pentraxin 3, Angiotensinogen, Follistatin, CF VII, Persephin, TRAIL R1, THAP11, CD200, CLEC-2, AMIGO, IGFBP-5, PON1, SOX7, GALNT10, Visfatin, Progranulin, PCSK2, GKN1, IL-18, Neprilysin, Stabilin-2, IL-17 RD, Albumin, Follistatin-like 1, MMP-10, FKBP51, LRRC4, Pref-1, Galectin-1, Troponin C, UNC5H3, FLRT2, CD314, Semaphorin 6B, Netrin-4, CD27 Ligand, IL-20 R beta, Semaphorin 6A, TSK, Cytokeratin-8, CHST3, Mcl-1, DPPIV, SREC-II, Norrin, JAM-C, Bcl-10, Wnt-4, LSECtin, Kell, TNF RI, PTP1B, htPAPP-A, IDO, PDGF-CC, Galanin, Activin A, TLR2, SCCA2, FABP1, eNOS, SHP-1, ICOS, ClqTNF9, MMP-1, TC-PTP, IL-24, gp130, C-myc, LILRB4, BMP-2, MIA, CD34, CD63, CD9, CD81, IFNab R2, Glypican 2, MSP R, DSCAM, Matriptase, KIR2DL3, CD30, Siglec-10, CLEC-1, TPP1, Ubiquitin+1, ANGPTL4, TWEAK R, Nidogen-1, CD2, Kallikrein 1, TSLP R, LAMP1, TROY, VCAM-1, Siglec-11, S100A1, PAR1, Thyroid Peroxidase, Aminopeptidase P2, IL-1 RI, ADAMS, OSM R beta, Thrombospondin-2, SMPD1, B2M, MFRP, LRP-6, ST3GAL1, NCAM-1 (CD56), Granzyme B, Adiponectin, IL-22BP, TPST2, PD-ECGF, LH, LEDGF, Cyr61, ULBP-3, IFNb, THSD1, FGF-23, LAMA4, Adipsin, AIF, SorCS2, SULT2A1, CD39L2, Insulin R, HIF-1 alpha, OX40 Ligand, Pax3, UCH-L3, cMASP3, Langerin, Desmin, SOX9, ST6GAL1, MEP1B, CD99-L2, Plexin A4, Semaphorin 4D, ROBO2, PDX-1, APRIL, Neurturin, Kremen-2, EMMPRIN, Activin RIB, Neuroligin 2, Epiregulin, CASA, MMP-12, GALNT2, CEACAM-5, VEGF R1, DSPG3, SorCS1, Matrilin-2, sFRP-3, p53, EphB3, NCK1, Semaphorin 7A, NKp80, Prolactin, Cystatin B, Sirtuin 1, FGF-16, FGF R5, NQO-1, Semaphorin 6D, FGF-3, GATA-4, VAP-A, CHST2, Pappalysin-2, Syndecan-3, Jagged 1, AKR1C4, Olfactomedin-2, Osteoadherin, NKp44, Thyroglobulin, IL-21R, Chemerin, EphA1, CD48, MICB, FGF-5, TRANCE, CES2, ULBP-1, Integrin alpha 5, VAMP-2, FLRG, Ret Midkine, CD73, TRACP, proGRP, Granzyme H, PRX2, p2′7, Siglec-6, Dectin-1, CD51, Notch-1, Calreticulin, DR3, DCTN1, CDC25B, Osteoactivin, ACE, CA125, HAO-1, PSMA1, FCRLB, BMP-9, CRIM1, LIF, SPINK1, EphB6, RGM-B, HS3ST1, ROR1, CMG-2, 4-1BB Ligand, L1CAM-2, p63, Cathepsin V, Testican 2, Glypican 5, CD6, Siglec-2, Legumain, PRELP, CES1, TAZ, NSE, TECK, HTRA2, HIF-1 beta, TAFA1, Podocalyxin, RalA, CRELD2, GRAP2, SP-D, BID, GFR alpha-2, Notch-3, VEGF R3, DLL4, TGFb2, LIGHT, XIAP, ST8SIA1, Cathepsin L, 6Ckine, MIS RI, Kallikrein 5, TGM3, FCAR, Contactin-2, CD83, IL-1 R3, SALM4, GBA3, ROBO4, OSCAR, VEGF, IGSF3, Biglycan, Neudesin, ILT4, uPAR, Axl, WIF-1, IL-7 R alpha, GPR56, CEACAM-3, MCEMP1, FABP2, Plexin B3, MEPE, Activin RIIA, ANG-2, Cochlin, Presenilin 1, NPTXR, SLAM, COMT, SPHK1, RBP4, Nectin-1, GUSB, Nidogen-2, IL-17F, SR-AI, TAFA2, N-Cadherin, IL-17B, IL-17 RC, MIP-3b, Cystatin C, Cystatin D, AMSH, FcERI, CLEC10A, HGF R, ANG-1, Prolactin R, FGF-20, CD28, Nogo-A, HSD17B1, IL-19, Enteropeptidase, Cathepsin E, TSLP, TCN2, GDF-15, Epimorphin, GRKS, PD-1, Serpin A4, ADAM23, NOV, Galectin-2, Neurexin 3 beta, TLR3, Sirtuin 2, Numb, IL-28 R alpha, IL-33, Lin28, FCRL1, KLF4, NKp30, Lymphotactin, Cystatin SN, JAM-A, Calreticulin-2, ErbB4, BMP-8, IL-27 Ra, Fas, IL-4 Ra, Kallikrein 14, Matrilin-3, Olig2, Kallikrein 12, CA13, IL-9, Nectin-3, MPIF-1, Cystatin S, ADA, IL-2 Rb, GFR alpha-1, Smad4, ICAM-1, MEF2C, TREM-1, L-Selectin, Hepsin, CD42b, MCSF, RANK, CHST4, CA8, FCRL3, ASAH2, CF XIV, PYY, HGF, I-TAC, Semaphorin 4C, SorCS3, Tie-1, IL-31 RA, Arginase 1, POGLUT1, IL-Ira, Podoplanin, TIM-3, CREG, CD300f, uPA, EphA2, LRRTM4, LIMPII, Tenascin R, CPE, PECAM-1, DNAM-1, DKK-1, OPG, CPB1, TSH, MMP-2, Siglec-9, ICAM-3, Cystatin SA, Galectin-4, Pepsinogen II, Desmoglein-3, Nectin-4, SCF, Serpin A5, PTH, FGF-19, MSP, IL-28A, FGF-12, METAP2, ASAHL, EDIL3, NTAL, EGF R, TAFAS, Galectin-9, vWF-A2, TACE, Activin RIM, Cathepsin S, LDL R, BMPR-IA, OX40, IL-13 R2, B7-H4, MMP-13, ANGPTL7, TRAIL R4, IGSF4B, Sirtuin 5, PEAR1, SH2D1A, Cerberus 1, GDF-11, Nrf2, TROP-2, NUDTS, ROR2, EphB4, Glypican 1, LAP(TGFb1), Gash, Contactin-1, IL-27, UNC5H4, ICAM-2, MBL, HS3ST3B1, RCOR1, IL-10 Rb, XEDAR, IL-22, PILR-alpha, NRG1-131, FABP4, RGM-A, RELT, TrkC, CSa, SREC-I, Nestin, TPO, ErbB3, Kirre13, FLRT1, Galectin-3, CXCL16, JAM-B, DR6, Nogo Receptor, TLR4, VEGF R2, Tie-2, IL-15 R, Caspr2, LTbR, LAMP, ALCAM, GLP-1, NG2, IL-22 R alpha 1, AMIGO2, HCC-1, TFPI-2, ULBP-2, Desmoglein 2, Aggrecan, Syntaxin 4, VAMP-1, Nectin-2, FGF-21, Flt-3, GFAP, TIM-1, Inhibin A, Cadherin-4, P1GF-2, Neurogranin, HE4, IL-23 R, Galectin-7, GALNT3, GITR L, CD14, R-Spondin 2, CK19, Cardiotrophin-1, TREML1, HAPLN1, CD27, ANG-4, Siglec-7, CD155, VEGF-C, TNF RII, PGRP-S, SDF-1a, PDGF-AB, GPVI, CD40, SCF R, Thrombospondin-5, IL-1 RII, Neuropilin-2, Cadherin-13, E-Selectin, GITR, WISP-1, Renin, AgRP, MDL-1, ROBO3, RANTES, Endocan, Granulysin, hCGb, Mesothelin, TLR1, TRAIL, MOG, DDR1, NGF R, TRAIL R3, Trypsin 3, ARSB, LIF R alpha, BAFF R, CD157, Granzyme A, 2B4, ESAM, IL-1 R4, CXCL14, IL-31, SIRP alpha, Uromodulin, CTRC, CEACAM-1, TARC, MIP-3a, SDF-1b, NKp46, MCP-3, IL-32 alpha, TGFb3 FOLR2, CD58, IL-23, CD36, TNFb, Shh-N, Ficolin-1, Reg4, ILT2, Mer, TREM-2, Flt-3L, CDS, IL-6, CD229, Insulin, Syntaxin 6, GRO, Bcl-w, Lipocalin-2, PDGF-AA, IL-2 Ra, Angiogenin, LYVE-1, CD4, RAGE, CDNF, Brevican, NAP-2, PU.1, EDAR, ADAMTS13, Kynureninase, PTH1R, IFN-gamma R1, CrkL, B7-1, PARC, Draxin, VE-Cadherin, Procalcitonin, SOX15, Kallikrein 11, BCMA, Dectin-2, EpCAM, HCC-4, TGFa, IP-10, BLAME, CILP-1, PIGF, LOX-1, MCP-2, Resistin, HVEM, ENPP-7, Syndecan-4, IL-2 Rg, MICA, Dopa Decarboxylase, NPDC-1, MCP-4, EG-VEGF, Glycoprotein V, Semaphorin 4G, IL-12p40, PSA-total, IL-15, MAP1D, C1q, TNF4, Dtk, Endoglin, ENA-78, Reg3A, MIP-1b, FGF-17, IL-6R, IL-8, Galectin-8, CA4, Cystatin E M, FUT8, B7-H3, GCP-2, CD40L, MDC, 4-1BB, HO-1, SOST, S100A13, Kallikrein 7, or IL-13.

84. The use of any one of claims 79 to 83, wherein the extracellular vesicles comprise one or more of the following nucleic acids: hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-1′7-5p, hsa-miR-181a-5p, hsa-miR-191-5p, hsa-miR-193a-5p, hsa-miR-193b-3p, hsa-miR-19′7-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, or hsa-miR-99b-5p.

85. The use of any one of claims 79 to 84 wherein the composition is produced by:
(a) culturing bone marrow-derived MSCs under the following conditions to produce an MSC conditioned media:
(i) oxygen tension below 5%; and
(ii) culture media having a pH below 7;
(b) harvesting the MSC conditioned media; and
(c) formulating the MSC conditioned media to produce the therapeutic MSC secretome composition, wherein the therapeutic MSC secretome composition comprises proteins and extracellular vesicles produced by the bone marrow-derived MSCs in step (a).

86. The use of claim 85, wherein the culture media is serum-free.

87. The use of claim 85 or 86, wherein the culture media has a glucose concentration below 4.5 g/L.

88. The use of any one of claims 85 to 87, wherein the subject has a decrease in a BPI score of at least 10%, at least 20%, at least 30%, or greater than 30% after administration of the therapeutic MSC secretome composition compared to before administration of the therapeutic MSC secretome composition.

89. The use of claim 88, wherein the subject has a decrease in a BPI pain interference score of at least 10%, at least 20%, at least 30%, or greater than 30% after administration of the therapeutic MSC secretome composition compared to before administration of the therapeutic MSC secretome composition.

90. The use of any one of claims 85 to 89, wherein the subject has an increase in a range of motion assessment score of at least 50%, at least 60%, at least 70%, at least 75%, at least 80%, at least 85%, at least 95%, or at least 100% after administration of the therapeutic MSC secretome composition compared to before administration of the therapeutic MSC secretome composition.

91. The use of any one of claims 85 to 90, wherein the subject has an increase in a Jamar dynamometer test assessment score after administration of the therapeutic MSC secretome composition as compared to before administration of the therapeutic MSC secretome composition.

92. The use of claim 91, wherein the increase in the Jamar dynamometer test assessment score is in an upper extremity.

93. The use of claim 91 or 92, wherein the subject has an equalization of grip strength after administration of the therapeutic MSC secretome composition.

94. The use of any one of claims 91 to 93, wherein the subject has an increase in grip strength of at least 5 kg, at least 10 kg, at least 15 kg, at or least 20 kg after administration of the therapeutic MSC secretome composition compared to before administration of the therapeutic MSC secretome composition.

95. The use of any one of claims 91 to 94, wherein the subject has an increase in grip strength of at least 50%, at least 75%, at least 100%, at least 125%, at least 150%, at least 175%, at least 200%, or at least 210% after administration of the therapeutic MSC secretome composition compared to before administration of the therapeutic MSC secretome composition.

96. The use of any one of claims 91 to 95, wherein the subject has an increase in grip strength in an affected limb.

97. The use of any one of claims 91 to 96, wherein the subject has an increase in grip strength in a control limb.

98. The use of any one of claims 79 to 97, wherein the subject has an improvement after administration of the therapeutic MSC secretome composition compared to before administration of the therapeutic MSC secretome composition in one or more of: limb range of motion, joint range of motion, sudomotor changes, edema, allodynia, and hypesthesia.

99. The use of any one of claims 79 to 98, wherein the subject has an improvement in an Short Form 36 Health Survey Questionnaire (SF-36) Questionnaire score after administration of the therapeutic MSC secretome composition compared to before administration of the therapeutic MSC secretome composition.

100. The use of claim 99, wherein the improvement in the SF-36 Questionnaire score comprises one or more of: an improvement in pain, an improvement in energy levels, an improvement in social functioning, an improvement in physical functioning, a positive trend in emotional wellbeing, and an improvement in general health.

101. The use of any one of claims 79 to 100, wherein the subject has chronic intractable pain of one or more upper or lower extremities.

102. The use of any one of claims 79 to 101, wherein the subject does not experience escalating or changing pain in the 30 days preceding administration of the therapeutic MSC secretome composition.

103. The use of any one of claims 79 to 102, wherein the subject experiences decreased pain flare-ups, decreased swelling, and/or decreased burning sensations in an affected limb after administration of the therapeutic MSC secretome composition compared to before administration of the therapeutic MSC secretome composition.

104. The use of any one of claims 79 to 103, wherein the administration of the therapeutic MSC secretome composition improves hair and/or nail growth in an affected limb of the subject.
